(19) United States
(12) Patent Application Publication
(10) Pub. No.: US 2010/0166704 A1
(43) Pub. Date: Jul. 1, 2010

(54) Methods and compositions for inhibition of viral replication

(76) Inventors: Margo A. Brinton, Decatur, GA (US); Mohamed M. Emara, Roswell, GA (US); Wei Li, Wayland, MA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(21) Appl. No.: 12/082,095

(22) Filed: Apr. 7, 2008

Related U.S. Application Data
(63) Continuation-in-part of application No. 10/654,273, filed on Sep. 2, 2003.
(60) Provisional application No. 60/910,611, filed on Apr. 6, 2007.
(60) Provisional application No. 60/407,105, filed on Aug. 30, 2002.

(51) Int. Cl. A61K 38/21; A61K 39/395; A61K 35/76; A61K 38/00; A61K 38/16; A61K 31/7088

(52) U.S. Cl. 424/85.4; 424/130.1; 424/93.6; 514/2; 514/12; 514/44 R

(57) ABSTRACT

The present invention is directed to methods and compositions that are effective in the inhibition of viral replication. In particular, the methods and compositions are effective at interfering with the activity of host cell proteins required in viral replication. For example, an embodiment of the invention is directed to inhibition of flavivirus replication wherein the replication is affected by changing the normal interactions of the host cell protein TIAR or TIA-1.

### RELATED APPLICATIONS

This application claims, under 35 U.S.C. §119(e), the benefit of U.S. Provisional Patent Application No. 60/910,611, filed on Apr. 6, 2007, and is a continuation-in-part of U.S. patent application Ser. No. 10/654,273, filed on Sep. 2, 2003, which claims, under 35 U.S.C. §119(e), the benefit of U.S. Provisional Patent Application No. 60/407,105, filed Aug. 30, 2002, each of which is herein incorporated in its entirety.

### GOVERNMENT LICENSE RIGHTS

This invention was made with U.S. Government support under Public Health Service Research Grant AI048088 from the National Institute of Allergy and Infectious Diseases of the National Institutes of Health. The government has certain rights in the invention.

### TECHNICAL FIELD

This invention is directed to methods and compositions for the inhibition of viral replication. In particular, the invention is directed to methods and compositions that interact with host proteins necessary for viral replication, or that interact with the viral nucleic acid to inhibit viral replication.

### BACKGROUND OF THE INVENTION

Viruses cause some of the most debilitating illnesses known in humans, animals and plants. Vaccination procedures have provided some relief for humans and animals from some of the more deadly viruses, such as smallpox, measles, influenza and poliovirus. However, many viruses still cause much human suffering, loss of work days, death to animals and destruction of plants. Unlike bacteria, viruses use the host's own cellular mechanisms to reproduce.

Increased globalization has resulted in the invasion of new territories by viruses that were previously found only in specific geographic locations. A well known example of this is the spread of human immunodeficiency virus around the world. A recent example is the spread of West Nile virus into and through the United States.

West Nile virus is a mosquito-borne virus that was first isolated in 1937 from the blood of a patient in the West Nile region of Uganda. It has been endemic in parts of Africa, the Middle East and India. Wild birds are the main reservoir hosts, with human and horses acting as incidental hosts with no role in virus transmission.

West Nile virus (WNV) was first detected in the Western hemisphere in 1999, in New York State, United States. The mode of introduction of WNV into the United States is not known, but phylogenic analysis of the envelope gene of a WNV isolate indicates it was closely related to a WNV isolate in Israel. WNV transmission reoccurred in New York during the summers of 2000 and 2001, and the virus has spread southward and westward in the United States. It is expected that the virus will continue to spread throughout the United States, Canada, the Caribbean, and Central and South America. Mosquitoes capable of transmitting WNV to susceptible birds exist in all of these regions.

The incidence of clinical disease among WNV-infected humans is low, though in recent outbreaks there has been an increase in the severity of disease among those that develop clinical symptoms. Fever is the most common symptom, and other symptoms include headache, muscle weakness, and disorientation. A few infected individuals develop encephalitis, meningoencephalitis, polio-like paralysis, Parkinson's disease-like symptoms, or hepatitis. Most infected persons show no sign of infection. It is thought that in the 1999 outbreak in the U.S., 1900 persons were infected. Sixty-two developed clinical disease and of these, seven died.

It is expected that microbial agents, such as viruses, will continue to be spread to new territories and that such agents will need to be identified and treatments provided to the unprotected populations. One method of protection that would not be dependent on specific viral identification would be to provide compositions that interact with host proteins that are commonly involved in the viral replication pathways of different related viruses, such as flaviviruses, to stop or interfere with viral replication.

What is needed are methods for identification of components of the viral replication cycle that can be interfered with or inhibited so that viral replication or the spread of infection in the host is interrupted, without harming the host, and the development of compositions that are effective in inhibiting or interfering with viral replication.

### SUMMARY OF THE INVENTION

The present invention is directed to methods and compositions for inhibiting viral replication. Methods are described herein that identify proteins or regions of the viral nucleic acid that are important in viral replication. In particular, flavivirus replication is described wherein host proteins are important in replication of the virus. Methods of the present invention also include inhibiting the interactions of host proteins with viral components or inhibiting or interfering with viral nucleic acids to inhibit viral replication. Compositions comprising compounds, including nucleic acid constructs or small molecules that inhibit such viral replication are also included in the present invention.

Such compositions are easily administered by oral, subcutaneous, intravenous, and routes known to those skilled in the art and can be given in dosages that are safe and provide inhibition of viral replication. The present invention provides methods of treating diseases, found in humans, animals, and plants mediated by viral infection, comprising administering compositions comprising anti-viral compounds in dosages effective to inhibit viral replication.

### BRIEF DESCRIPTION OF THE FIGURES

FIG. 1A-B show polyacrylamide gels showing gel mobility shift experiment results.

FIG. 2 shows immunoprecipitation of UV-induced cross-linking RNA-protein complexes by protein specific antibodies.

FIG. 3 A-D show analyses of the specificity of the interactions between the WNV 3′(−) SL RNA and recombinant TIAR or TIA-1 proteins.

FIG. 4 A-D show quantification of the RNA-protein interactions.

FIG. 5 A-F are graphs showing the growth of virus in TIAR or TIA-1-knockout cell lines.

FIG. 6 A-F show Western blot analyses of the amounts of TIAR and TIA-1 proteins in various cell lines.

FIG. 7 is a schematic of host proteins interacting with WNV RNA.

FIG. 8 A-B are graphs of the colocalization of TIA-1 and TIAR with WNV proteins in infected BHK cells.

FIG. 9 is a immunoprecipitation and Western blot demonstrating the interaction of TIA-1 and TIAR with WNV replication complex components.

FIG. 10 A-B are a graph and Western blot, respectively, of stress granule formation during WNV infection.

FIG. 11 A-B are graphs of processing body formation during WNV infection.

FIG. 12 A-B are a graph and a Western blot, respectively, of stress granule formation during DV infection.

FIG. 13 A-B are graphs of processing body assembly during DV infection.

### DETAILED DESCRIPTION

The present invention is directed to methods and compositions that are effective in inhibiting viral replication. In particular, the present invention is directed to methods for identifying components of the viral replication cycle that are necessary for replication, such as proteins, and testing for compounds that are effective in the inhibition of these components. Inhibition of viral replication leads to little or no infected state in the organism, or reduces or terminates the infection in the organism.

Compositions and methods for the treatment of viral diseases that are mediated by inhibition of viral replication are also provided. Particularly, methods and compositions of the present invention are directed to inhibition of the activity of host cell proteins that are required for replication of viruses. Methods and compositions of the present invention are also directed to inhibiting or interfering with the viral nucleic acids to inhibit viral replication. Methods and compositions of the present invention are also directed to inhibiting or interfering with the viral proteins to inhibit viral replication. Methods for the identification of cellular factors that are required to complete various steps of a virus lifecycle are also provided.

For example, one family of viruses that creates debilitating and deadly disease in humans and animals is Flaviviridae. This family consists of three genera, one of which is the genus Flavivirus. Members of the Flavivirus genus include but are not limited to Gadgets Gully virus (GGYV), Kadam virus (KADV), Kyasanur Forest disease virus (KFDV), Langat virus (LGTV), Omsk hemorrhagic fever virus (OHFV), Powassan virus (POWV), Royal Farm virus (RFV), Tick-borne encephalitis virus (TBEV), Louping ill virus (LIV), Meaban virus (MEAV), Saumarez Reef virus (SREV), Tyuleniy virus (TYUV), Aroa virus (AROAV), Dengue virus (DV), Kedougou virus (KEDV), Cacipacore virus (CPCV), Koutango virus (KOUV), Japanese encephalitis virus (JEV), Murray Valley encephalitis virus (MVEV), St. Louis encephalitis virus (SLEV), Usutu virus (USUV), West Nile virus (WNV), Yaounde virus (YAOV), Kokobera virus (KOKV), Bagaza virus (BAGV), Ilheus virus (ILHV), Israel turkey meningoencephalomyelitis virus (ITV), Ntaya virus (NTAV), Tembusu virus (TMUV), Zika virus (ZIKV), Banzi virus (BANV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), Yellow fever virus (YFV), Entebbe bat virus (ENTV), Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), and Rio Bravo virus (RBV), among others. Flaviviruses are generally transmitted between bird and mammalian hosts via mosquitoes or ticks. Flaviviruses, such as Dengue virus (DV), Japanese encephalitis virus, West Nile virus (WNV), yellow fever virus, St. Louis encephalitis virus, Murray Valley encephalitis virus, and tick-borne encephalitis virus, can sometimes cause severe disease in infected humans.

The genomes of flaviviruses are single-stranded, positive-polarity RNAs of approximately 11 kb containing a single open reading frame that encodes a single large polyprotein that is post-translationally processed by viral and cellular proteases into three structural proteins (capsid, membrane, and envelope) and seven non-structural proteins (NS1, NS2a, NS2b, NS3, NS4a, NS4b, and NS5). The C-terminal portion of NS5 is the RNA dependent RNA polymerase (RdRp), while the N-terminal part is a methyltransferase involved in RNA capping. The N-terminus of NS3 has serine protease activity, while the C-terminus contains helicase, NTPase, and triphosphatase activities. The NS3, NS5, and NS1 proteins as well as the hydrophobic NS2a, NS2b, NS4a, and NS4b proteins were previously shown to be components of membrane bound viral RNA replication complexes located in the perinuclear region of infected cells. During the flavivirus replication cycle, which takes place in the perinuclear region of the cytoplasm of infected cells, the genomic RNA (plus strand RNA) serves as the only viral mRNA and is also the template for transcription of the complementary minus-strand RNA. The minus-strand RNA in turn serves as a template for the synthesis of genomic RNA. Plus-strand RNA synthesis is 10 to 100 times more efficient than minus-strand synthesis.

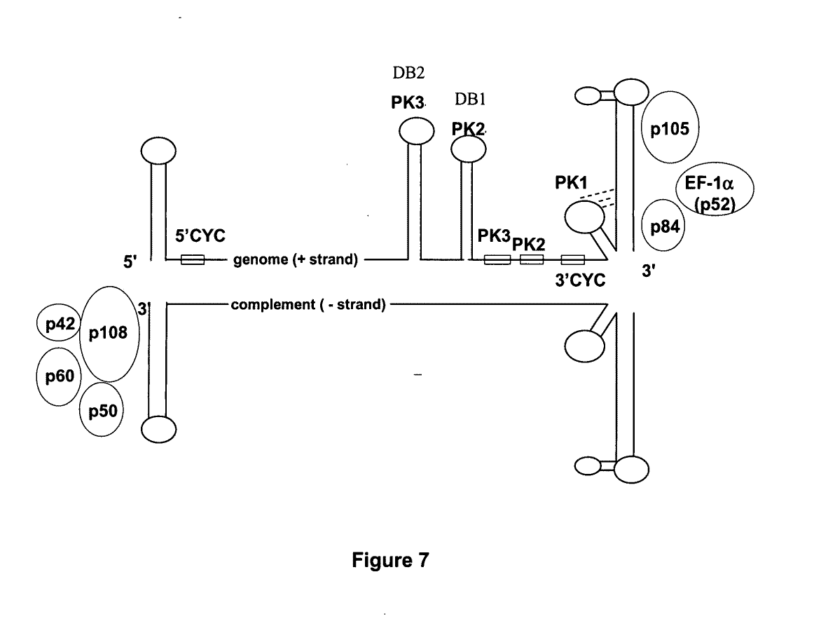

The non-coding regions (NCRs) of the flavivirus genome contain terminal RNA structures that are conserved between divergent flaviviruses even though only short sequences in these regions are conserved. The terminal RNA structures located at the 3′ ends of the genome and complementary minus strand RNAs differ from each other in shape and size. Deletion or mutation of either 3′ terminal structure in flavivirus infectious clones resulted in no progeny virus production and indicated that these regions were essential for virus replication. However, specific cis-acting signal sequences within these structures have not yet been mapped nor functionally analyzed. The WNV 3′ terminal RNA plus strand and minus strand RNA structures have previously been reported to bind specifically to different sets of cell proteins (FIG. 7).

An aspect of the present invention comprises methods and compositions that are effective in modifying the activity or interactions of components or proteins involved in the initiation and regulation of nascent genome RNA synthesis from the minus strand template, nascent minus strand RNA from the genome template, as well as translation of the viral RNA into protein and production of mature proteins for the virus. As used herein, inhibition or interference in viral replication means any change in the rate of viral replication or in the amount of viral components made after infection of a cell by a virus. The change is preferably a decrease in rate or amount of replication, though inhibiting or interfering at one step of the replication pathway may lead to an increase in the precursors necessary for that step.

The presence in solution of the 3′ terminal structure of the WNV genomic (plus strand) RNA [WNV 3′ (+) SL RNA] was previously confirmed by RNase structure probing. Three RNA-protein complexes (RPCs) were detected by gel shift mobility assays performed with Baby Hamster Kidney cell (BHK) cytoplasmic extracts and the WNV 3′ (−) SL (stem loop) RNA probe. The same pattern of RNA-protein complexes was observed when WNV-infected or uninfected BHK S100 cytoplasmic cell extracts were used, suggesting that the proteins in these complexes were cellular proteins. The results of UV-induced crosslinking and Northwestern blotting studies indicated that the molecular masses of the RNA binding proteins in these complexes were 52, 84, and 105 kDa (FIG. 7). The specificity of these RNA-protein interactions was demonstrated by competition gel mobility shift and competition UV-induced cross-linking assays. The p52 protein was identified as EF-1α.

An aspect of the present invention comprises compositions that are effective in inhibiting or interfering with the interactions of host cell proteins, p52, p84 and p105, that are involved in viral replication and particularly with the interactions of these proteins with the WNV 3′ (+) SL RNA. Methods of assaying for such compositions comprise adding compositions to cells infected with WNV, or cell-free systems, and measuring the reduction in viral replication, compared to infected cells or cell-free systems without the composition. An embodiment of the invention comprises adding a composition comprising a nucleic acid fragment that mimics the 3′ (+) SL RNA in an amount effective to inhibit or interfere with viral replication.

The presence in solution of the 3′ terminal structure of the WNV minus-strand RNA [WNV 3′ (−) SL RNA] was previously confirmed by RNase structure probing. Three RNA-protein complexes (RPCs) were detected by gel shift mobility assays performed with BHK cytoplasmic extracts and the WNV 3′ (−) SL RNA probe. The same pattern of RNA-protein complexes was observed when WNV-infected or uninfected BHK S100 cytoplasmic cell extracts were used, suggesting that the proteins in these complexes were cellular proteins. UV-induced crosslinking studies indicated that the molecular masses of the RNA binding proteins in these complexes were 42, 50, 60, and 108 kDa (FIG. 7). The specificity of these RNA-protein interactions was demonstrated by competition gel mobility shift and competition UV-induced cross-linking assays. p42 has been identified as TIAR or TIA-1.

FIG. 7 is a schematic drawing showing the conserved structures and sequences in WNV RNAs and the cell proteins that bind specifically to the 3′ terminal regions of these RNAs. The top line is the WNV genomic RNA, and the bottom line is the complementary minus strand RNA. (PK—pseudoknot; DB—dumbbell shaped RNA structures) Cell proteins are indicated by circles. Competition gel mobility shift data suggest that p108 and p105 may be the same protein.

An aspect of the present invention comprises compositions that are effective in inhibiting or interfering with the interactions of host cell proteins, particularly TIA-1 and TIAR, that are involved in viral replication of flaviviruses. An aspect of the present invention further comprises compositions that are effective in inhibiting or interfering with the interactions of host cell proteins, particularly TIA-1 and TIAR, that are involved in viral replication of flaviviruses and particularly with the interactions of these proteins with the secondary structure of (+) strand and/or (−) strand flavivirus RNA. Another aspect of the present invention comprises compositions that are effective in inhibiting or interfering with the interactions of TIA-1 and TIAR that are involved in viral replication of flaviviruses and particularly with the interactions of these proteins with the stem loop structures of the (−) strand flavivirus RNA. Methods and compositions for the inhibition of flavivirus replication are further described in U.S. patent application Ser. No. 11/879,493, which is herein incorporated by reference in its entirety.

Methods of assaying for such compositions comprise adding the composition to be tested to a first set of cells, wherein the composition is added prior to, simultaneously with, or after, infection with a flavivirus. In a second set of cells of the same type as the first set of cells, the infection is allowed to proceed as normal to serve as a control. Other sets of cells may be used for other test or control conditions. As used herein wherein it is taught that the composition is added to cells infected with a flavivirus, it is intended that the composition may be added before the viral infection is initiated, or added when the viral infection is initiated by addition of virions to the cells, or added at some time point after initiation of viral infection.

Methods of assaying for such compositions comprise adding compositions to cells infected with a flavivirus, or cell-free systems, and measuring the reduction in viral replication, compared to infected cells or cell-free systems without the composition. An embodiment of the invention comprises adding a composition comprising a nucleic acid fragment that mimics the 3′ (−) SL RNA of a flavivirus in an amount effective to inhibit or interfere with viral replication.

An aspect of the present invention comprises compositions that are effective in inhibiting or interfering with the interactions of host cell proteins, particularly p42 (TIAR and TIA-1), p50, p60, and p108, that are involved in viral replication and particularly with the interactions of these proteins with the WNV 3′ (−) SL RNA. Methods of assaying for such compositions comprise adding compositions to cells infected with WNV, or cell-free systems, and measuring the reduction in viral replication, compared to infected cells or cell-free systems without the composition. An embodiment of the invention comprises adding a composition comprising a nucleic acid fragment that mimics the 3′ (−) SL RNA in an amount effective to inhibit or interfere with viral replication.

An aspect of the present invention comprises compositions that are effective in inhibiting or interfering with the interactions of host cell proteins, particularly TIAR, TIA-1, p50, p60, and p108 that are involved in viral replication and particularly with the interactions of these proteins with the viral nucleic acids of Dengue virus, and more particularly the 3′ (−) SL RNA. Methods of assaying for such compositions comprise adding compositions to cells infected with a flavivirus, for example but not limited to WNV or DV, or cell-free systems, and measuring the reduction in viral replication, compared to infected cells or cell-free systems without the composition. An embodiment of the invention comprises adding a composition comprising a nucleic acid fragment that mimics the 3′ (−) SL RNA in an amount effective to inhibit or interfere with viral replication.

An embodiment of the present invention comprises methods for identification of one of the WNV 3′ (−) SL RNA-binding proteins, p42, as T-cell intracellular antigen-related (TIAR) protein. The closely related protein, T-cell intracellular antigen-1 (TIA-1), was also shown to bind specifically to the WNV 3′ (−) SL RNA. Results from WNV growth studies in TIAR-knockout and TIA-1-knockout cells show these cell proteins are important in flavivirus replication. Such knock-out cells are also useful in assays for determining compositions that are effective in inhibiting flavivirus replication. For example, viral infections in knock-out cells for particular host proteins are useful for confirming the effects of selected compositions that are specific for interfering with one or more host proteins involved in viral replication. An embodiment of an assay comprises infecting knock-out cells, for example, cells lacking TIAR, with WNV, adding the composition being tested to one set of cells and not adding the composition to the control set of cells, and comparing the viral replication in the set of knock-out cells with the composition to the control set. Initial comparisons would be made in infected cells that were normal for the protein lacking in the knock-out cells.

An aspect of a method of the present invention comprises inhibiting replication of a virus, comprising, administering a composition capable of inhibiting at least one host cell protein needed for replication of a virus, wherein the virus is a flavivirus, wherein the flavivirus includes but is not limited to West Nile virus and Dengue virus. Methods include inhibition of at least one host cell, wherein the at least one host cell protein includes but is not limited to TIAR, TIA-1, p 52, p84, p105, p108, p42, p50, or p60. Compositions used in such methods include composition comprising a nucleic acid construct, and include nucleic acid constructs that mimic the 3′ end of the negative strand nucleic acid of a flavivirus or that mimic the 3′ end of the plus strand nucleic acid of a flavivirus. As used herein, mimicking a nucleic acid means having a nucleotide sequence that is identical to the nucleic acid or has sufficient sequence homology to the nucleic acid such that the mimicking nucleic acid has the same protein interactions or nucleic acid function as the original nucleic acid. Mimicking includes nucleic acid sequences or other molecules or synthetic materials that have the same structure as the original nucleic acid. The original nucleic acid, the one that is being mimicked, can be a fragment of a nucleic acid that includes the end nucleotides or any polynucleotide found between the ends. The original nucleic acid may comprise the sequence of known organisms, or have homology to known organism sequences, or may comprise derived sequences. For example, the nucleic acid sequence may be the sequence of a flavivirus, for example but not limited to West Nile virus or Dengue virus. The compositions may comprise a small organic molecule, antibody, peptide, peptoid, or polynucleotide.

For example, a nucleic acid mimic may have the sequence of nucleotides 1-400 of the 3′ end of a minus strand of a flavivirus, or nucleotides 1-200, or nucleotides 1-100, or nucleotides 1-78, or nucleotides 1-50, or nucleotides 1-25, of the minus strand of a flavivirus. A nucleic acid mimic may have the sequence of nucleotides 50-100 of the 3′ end of the minus strand of a flavivirus, the sequence of nucleotides 10-100, or nucleotides 1-80, or a sequence found on the 3′ end of the (−) strand that will bind host cell proteins such as TIA-1 or TIAR.

Another aspect of a method of the present invention comprises inhibiting replication of a virus, comprising, administering a composition capable of inhibiting the activity or binding of at least one host cell protein needed for replication of a virus, wherein the virus is a flavivirus, wherein the flavivirus includes but is not limited to Dengue virus or West Nile virus. Methods include inhibition of at least one host cell, wherein the at least one host cell protein includes TIAR, TIA-1, p52, p84, p105, p108, p42, p50, or p60. Compositions used in such methods include composition comprising a peptide construct, and include peptide constructs that mimic one or more regions or domains of TIAR, TIA-1, p52, p84, p105, p108, p42, p50, p60, or combinations thereof that interact with the 3′ end of the negative strand nucleic acid of a flavivirus or the 3′ end of the plus strand nucleic acid of a flavivirus. As used herein, mimicking a peptide means having an amino acid sequence that is identical to the amino acid sequence or has sufficient sequence homology to protein of interest such that the mimicking peptide has the same or sufficiently similar protein interactions or function as the nascent protein. Mimicking includes amino acid sequences, peptides, or other molecules or synthetic materials that have the same structure or function as the protein of interest or similar binding characteristics. The protein of interest, the one that is being mimicked, can be a fragment or a segment of a protein. The protein of interest may comprise the sequence of known organisms, or have homology to known organism sequences, or may comprise derived sequences. For example, the amino acid sequence may be the sequence of an animal, particularly a human. The compositions may comprise a small organic molecule, antibody, peptide, peptoid, or polynucleotide.

Embodiments of the present invention comprise assays for determining compositions that are effective in inhibiting or interfering with viral replication. One assay for determining compositions that inhibit viral replication, comprises, a) adding a composition to cells infected with a virus, and b) comparing the change in viral replication in the cells of a) to control cells infected with the virus. The change in viral replication can be any measureable change in viral replication including, but not limited to, a change in the rate of replication of the virus or a change in the amount of viral components synthesized. One embodiment comprises assays that determine compositions that are effective in inhibiting flavivirus replication, particularly West Nile virus. Another embodiment comprises assays wherein the cells are knockout cells, such as the cells described herein. Knockout cells can comprise cells lacking one or more nucleic acid sequences or proteins, particularly host cell proteins including but not limited to, TIAR, TIA-1, p52, p84, p105, p108, p42, p50, or p60.

In one embodiment of the present invention, an assay for determining compositions that inhibit viral replication of flaviviruses comprises: a) adding a composition to one set of control cells and a set of cells to be infected with a flavivirus (the composition may be added to the cells before or after viral infection); b) comparing the change in flavivirus replication of the cells of a) to cells infected with the flavivirus in the absence of the composition; c) comparing the change in flavivirus replication of the cells of a) to knockout cells infected with a flavivirus; wherein the knockout cells have a deletion of at least one protein, wherein the at least one protein is TIAR, TIA-1, p50, p60, p108, p52, p84, or p105; and d) determining whether the composition of a) changes the replication of the flavivirus. For example, the assay may be used for determining compositions that inhibit viral replication of flaviviruses, for example but not limited to West Nile virus or Dengue virus.

In another embodiment of the present invention, an assay for determining compositions that inhibit viral replication comprises: a) adding a composition to one set of control cells and to a set of cells to be infected with a flavivirus (the composition may be added to the cells before or after viral infection); b) exposing the cells to oxidative stress; c) comparing the number of stress granules of the cells of a) to control cells infected with the flavivirus in the absence of the composition; d) comparing the number of stress granules of a) to control uninfected cells exposed to oxidative stress; e) determining whether the composition of a) changes the number of stress granules. For example, the assay may be used for determining compositions that inhibit flavivirus replication, wherein the flavivirus is West Nile virus or Dengue virus.

In yet another embodiment of the present invention, an assay for determining compositions that inhibit viral replication comprises: a) adding a composition to cells infected with a flavivirus; b) exposing the cells to oxidative stress; c) comparing the number of processing bodies of the cells of a) to control cells infected with the flavivirus in the absence of the composition; d) comparing the number of processing bodies of a) to control uninfected cells exposed to oxidative stress; e) determining whether the composition of a) changes the number of processing bodies. In another embodiment of the present invention, the assay may be used for determining compositions that inhibit flavivirus replication, wherein the flavivirus is West Nile virus or Dengue virus.

Methods of the present invention also comprise methods for treating a viral infection, comprising, administering to a human or animal having a viral infection, a composition that alters the interaction of one or more host cell proteins with a viral nucleic acid in an amount effective to inhibit or interfere with viral replication. The compositions comprise those taught herein, including but not limited to, a nucleic acid construct, a small molecule, an antibody, a peptide, peptoid or polynucleotide. The host cell proteins affected include but are not limited to, TIAR, TIA-1, p52, p84, p105, p108, p42, p50, or p60. Treatments include treatment of infection of individuals with flaviviruses, particularly West Nile virus and Dengue virus.

Using methods disclosed herein, p42, one of the four cell proteins previously reported to bind specifically to the WNV 3′ (−) SL RNA, has been identified as TIAR and TIA-1. The methods can also be used to identify the other host cells involved in viral replication. It is the inventor's novel finding of the identification of a host protein that interacts specifically with the 3′ SL of a flavivirus minus-strand RNA, the site of initiation for nascent genome RNA synthesis. TIA-1 and TIAR are closely related, multifunctional, RNA-binding proteins that have at least partial redundancy in their cellular functions. The data herein shows that the binding of TIAR to the WNV 3′ (−) SL RNA is functionally important for viral replication.

TIAR and TIA-1 are evolutionarily conserved proteins; homologs in different mammalian species share 96% (TIA-1) and 99% (TIAR) identity, while homologs in divergent species such as Drosophila and Caenorhabditis elegans each share about 46% amino acid identity with human TIA-1 and TIAR. Because flaviviruses replicate efficiently in a large number of divergent host species and cycle between invertebrate and vertebrate hosts during their natural transmission cycles, it is expected that these viruses would need to interact with evolutionarily conserved cell proteins to replicate efficiently in different hosts. TIAR and TIA-1 proteins were initially discovered in T cells, hence their name, but have since been found to be expressed in good quantities in many tissues including brain, spleen and macrophages, which are sites of flaviviruses replication in vivo.

The present invention is not limited to inhibition or interference with just flavivirus replication, but contemplates the inhibition or interference of other viruses that use host proteins, particularly host proteins that bind to nucleic acids, more particularly host proteins that bind to viral RNAs, and are also not limited to interference or inhibition in any particular species of cells. Thus for example, for proteins that are conserved among species, compositions that are effective in inhibiting the activity of one or both TIAR or TIA-1 of one host species, such as human cells, are effective in inhibiting the replication of the viruses requiring TIAR or TIA-1 in other host species, such as birds or insects

Both TIAR and TIA-1 shuttle between the nucleus and the cytoplasm in viable cells. Flaviviruses replicate exclusively in the cytoplasm. Interestingly, the level of TIAR in the cytoplasm of BHK cells is about 10 times higher than in several mouse embryo fibroblast cell lines and WNV grows to about 10 times higher titers (peak titer of about 10^{7.5} PFU/ml) in BHK cells than in the mouse cell lines (peak titer of about 10^{6.5} PFU/ml).

In selection/amplification experiments with pools of randomized RNAs, both TIAR and TIA-1 bound with high affinity to RNAs that contained one or more short sequences of poly U with a dissociation constant of about 2×10⁻⁸M. Replacement of the Us in these RNAs with Cs eliminated the protein-RNA interaction. Although both proteins selected RNAs containing stretches of Us, the RNA sequences selected by TIA-1 were not identical to those selected by TIAR. The RNA recognition motif 2 (RRM2) domains in both proteins mediated specific binding to uridylate-rich RNAs. However, the presence of the other two RRM domains increased the affinity of the interaction of the proteins with the U-rich RNAs.

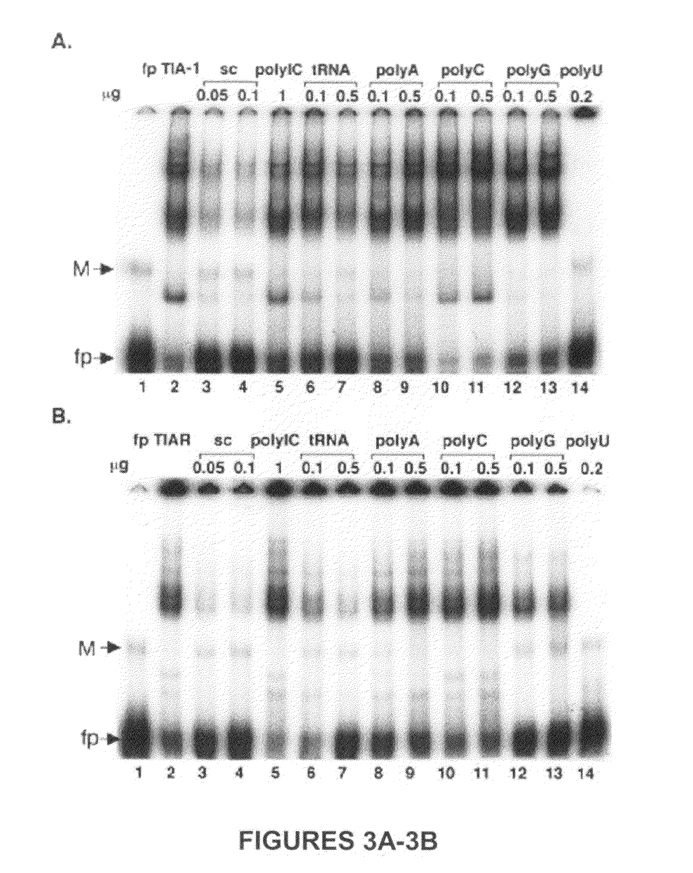

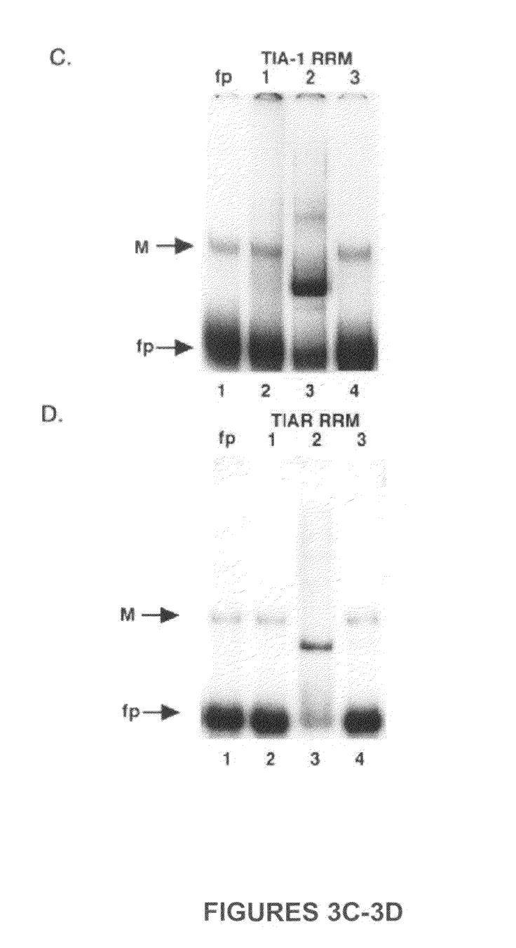

In the 3′ NCR of the TNF-α′ mRNA, a large fragment of AU-rich sequence containing clustered AUUUA pentamers was required for TIAR and TIA-1 binding. The data presented here indicate that both TIAR and TIA-1 can also bind specifically to the WNV 3′ (−) SL RNA and that the RRM2 domain mediates this interaction. Since poly U competed efficiently with the WNV 3′ (−) SL RNA in the competition gel-mobility shift assays (FIG. 3), it is expected that the viral sequence(s) recognized by TIAR and TIA-1 contains Us. Although the WNV 3′ (−) SL RNA is not AU-rich, two of the single stranded loops in this structure contain the sequences, UAAU and UUAAU. These sequences are conserved in single stranded loops in the SLs of other mosquito borne flaviviruses. Mapping studies are underway to identify the individual nucleotides in the WNV 3′ (−) RNA required for binding by each of these proteins.

The observed dissociation constant (K_{d}) for the interaction between TIAR RRM2 and the WNV 3′ (−) SL RNA was 1.5×10⁻⁸ M, which is similar to the K_{d} reported for the interaction between TIAR and U-rich synthetic RNA sequences (and also for other functionally relevant RNA-protein interactions, such as the interaction between the cellular U1A protein and the U1-RNA). Interestingly, the binding activity of TIA-1 (K_{d} of 1×10⁻⁷ M) for the WNV 3′ (−) SL RNA was about 10 times lower than that of TIAR. Comparison of the RRM2 domain sequences of TIA-1 and TIAR indicate that they differ at eight amino acid residues and that TIAR also contains an eleven amino acid deletion at the beginning of RRM2. The ten fold lower binding activity of TIA-1 for the WNV 3′ (−) SL RNA would be expected to result in TIAR out-competing TIA-1 for binding to the viral RNA affinity column and would reduce the likelihood of detecting unique TIA-1 peptides in the protein eluted from the viral RNA affinity column.

A number of cellular functions have been attributed to the RNA binding properties of TIA-1 and TIAR. Both TIAR and TIA-1 regulate the generalized translational arrest that occurs following an environmental stress. Stress-induced phosphorylation of the translation initiation factor eIF-2a is followed by recruitment of poly (A)⁺ RNA into cytoplasmic stress granules by TIAR and TIA-1. Stress granules and polysomes appear to be in equilibrium in cells. TIA-1 and TIAR also function as specific translational silencers. For example, TNF-α translation is blocked by the binding of TIAR and TIA-1 to specific U-rich sequences in the 3′ NCR of this mRNA. Upon stimulation with lipopolysaccharides (LPS), this translational repression is overcome by the binding of an additional protein, p55, to the 3′ NCR of the TNF-α mRNA. TIA-1 and TIAR have recently been shown to function as alternative splicing regulators by binding to specific U-rich intron (IAS1) sequences adjacent to cryptic 5′ splice sites and enhancing the use of these 5′ splice sites. Such intron sequences exist in a subset of pre-mRNAs, including those of TIA-1 and TIAR, and it is thought that both proteins can regulate their own expression at the level of splicing as well as the expression of some other proteins. The yeast protein, Nam8p, a component of the U1 snRNP, is distantly related to TIA-1 and TIAR. It is interesting to note that even though the majority of the known cellular functions of TIAR and TIA-1 involve interactions with cellular mRNAs, it is the 3′ terminal region of the WNV minus strand, not the positive strand genome, that interacts with these proteins.

Although both TIAR and TIA-1 have previously been implicated as effectors of apoptotic cell death, the specific roles of these proteins in apoptosis have not as yet been delineated. Introduction of purified TIAR or TIA-1 into the cytoplasm of thymocytes permeabilized with digitonin resulted in fragmentation of genomic DNA into nucleosome-sized oligomers. Increased amounts of TIAR were translocated from the nucleus to the cytoplasm in response to exogenous triggers of apoptosis. TIA-1 was shown to be phosphorylated by a serine/threonine kinase activated during Fas-mediated apoptosis. Although not rigorously tested, no evidence of apoptosis was observed when rodent cells infected with WNV were examined at intervals up to 32 hr post infection after fixation and nuclear staining with Hoerchst dye (data not shown). A study with WNV indicated that apoptosis occurred by 72 hr in infected human mononuclear (K562) cells and mouse neuroblastoma (Neuro 2a) cells via the BAX pathway.

Both TIAR and TIA-1 appear to play important roles in embryo development. However, the specific functions of these proteins during development are not known. It was not possible to produce double knock-out mice because of lethality prior to embryonic day 8.

Interestingly, of the five types of the viruses tested in the TIAR and TIA-1 knock-out cells, only the growth of the flavivirus, WNV, was decreased in cells lacking TIAR. In contrast, the growth of the four other types of viruses was more efficient in one or both types of knock-out cells as compared to that in wild type cells. These data suggest that in the wild type cells, one or both of these proteins have a negative effect on the production of these viruses. However, the negative effect that the loss of TIAR and to a lesser extent the loss of TIA-1 have on WNV replication suggests that these proteins provide a necessary function for WNV during its replication cycle.

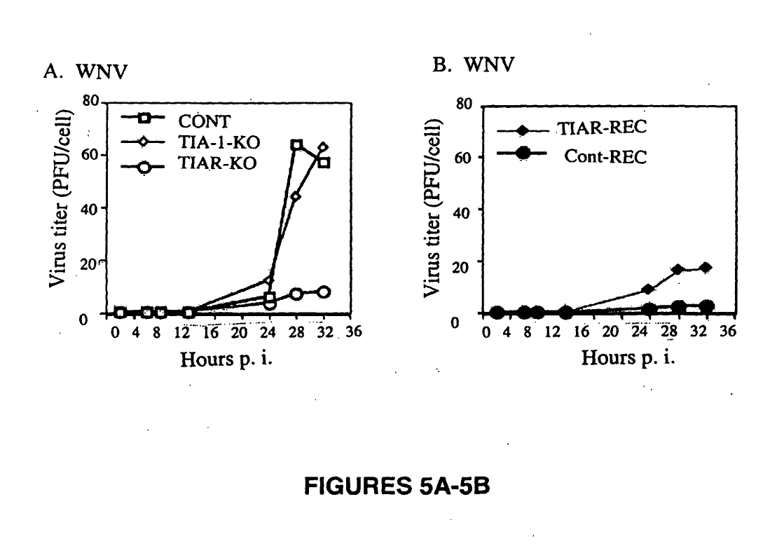

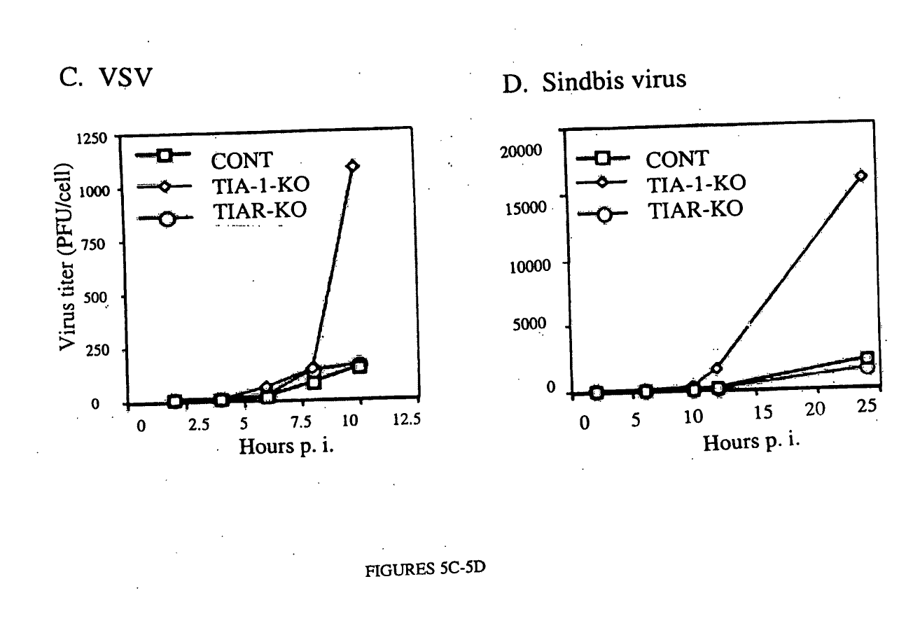

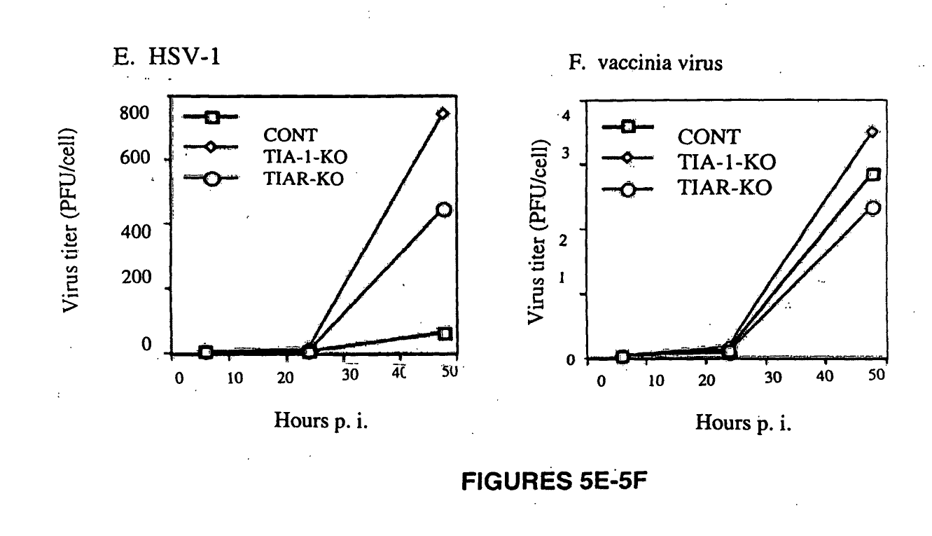

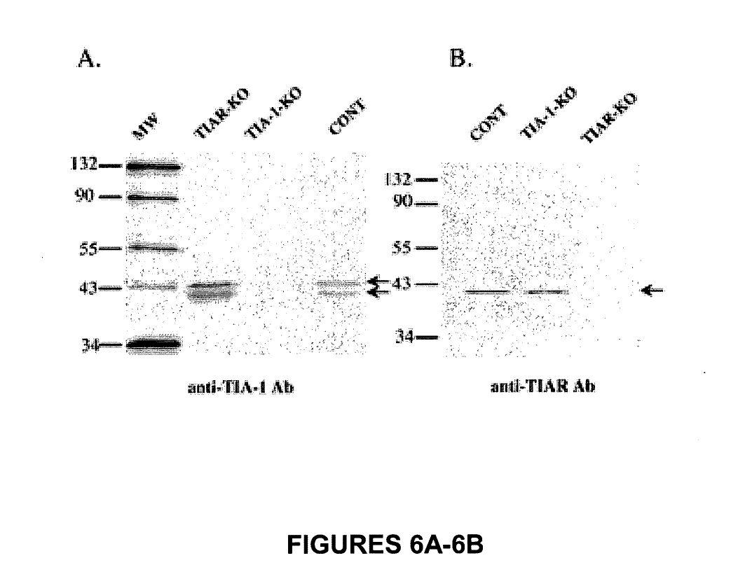

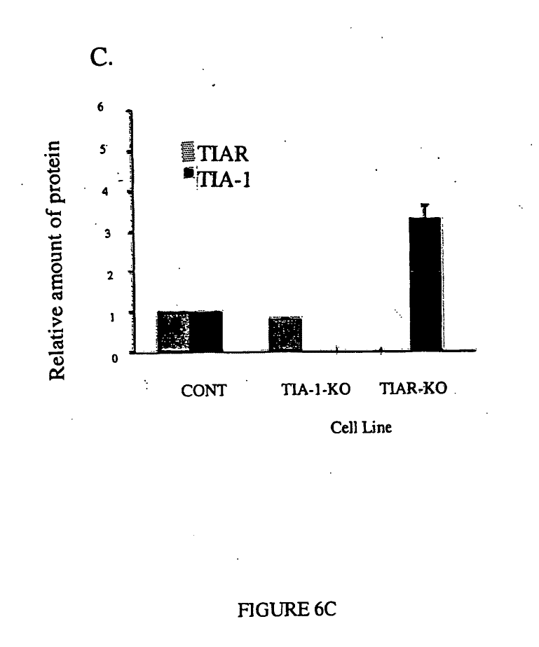

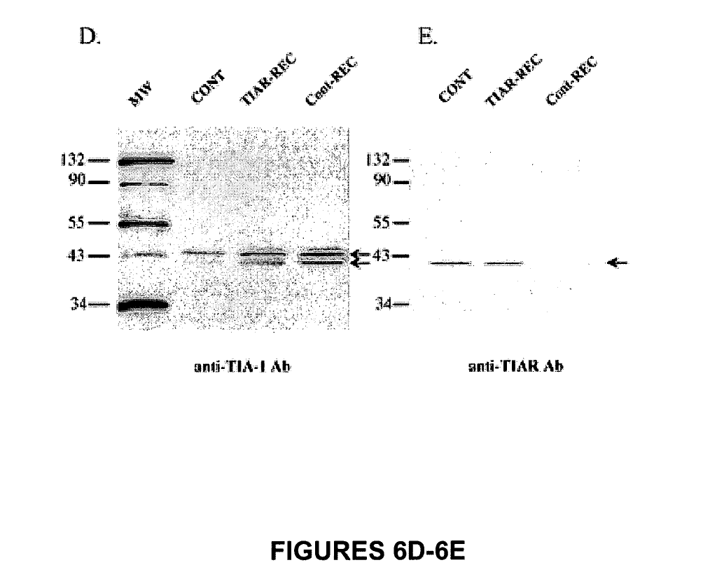

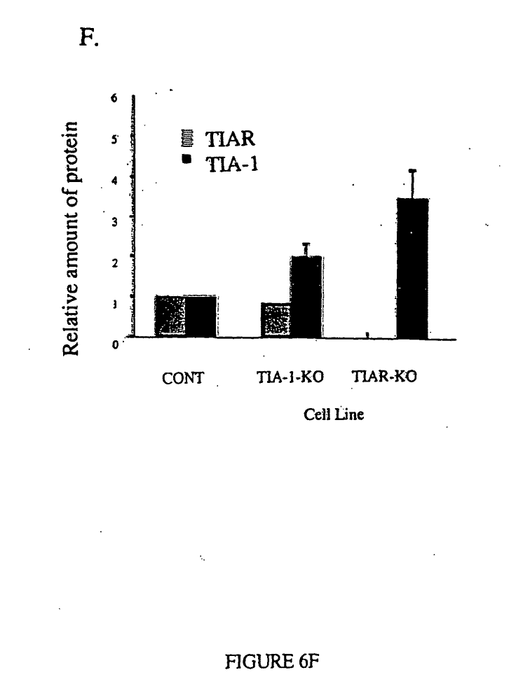

Though not wishing to be bound by any particular theory, one possible explanation for why the growth of WNV was not reduced to a greater extent in TIAR-knockout cells could be that the TIA-1 protein, which is present in increased amounts in the TIAR-knockout cells (FIG. 6), can substitute for TIAR by providing the function needed by the WNV. However, WNV replication in cells lacking TIAR was never as efficient as when TIAR was present. Also, although the efficiency of virus replication increased when TIAR-knockout cells were reconstituted with vector expressed TIAR (FIGS. 5 and 6), neither the amount of TIAR nor WNV replication reached wild type levels in these reconstituted cells.

The only known function of the flavivirus minus strand RNA is as a template for the synthesis of nascent genomic RNA. Specific binding of TIA-1 and TIAR to the 3′ terminus of the viral minus strand RNA template appears to play a positive role in virus replication. Possible functions of this interaction include assisting in the formation or stabilization the 3′ terminal (−) SL and/or in the recognition of the minus template by the polymerase.

Flavivirus infections do not shut off host cell translation and flaviviral nonstructural proteins and dsRNA (indicative of viral replication intermediates) have been co-localized to redistributed endoplasmic reticulum, trans-Golgi and intermediate compartment membranes. If the binding of TIA-1 and TIAR to viral minus strand RNAs in replicative intermediates results in their co-localization with stress granules, this would provide an environment in which the translation of growing nascent viral plus strand RNAs would be inhibited. Alternatively, the binding of TIA-1 and TIAR by the viral RNA could keep it from caring out its normal cell functions during stress.

Though not wishing to be bound by any particular theory, it is currently believed that eukaryotic cells respond to environmental stresses, such as oxidative stress, heat shock, UV irradiation, and endoplasmic reticulum (ER) stress, and some viral infections, by altering the protein expression machinery. The expression of proteins responsible for damage repair is increased, whereas translation of constitutively expressed proteins is aborted via redirection of these mRNAs from polysomes to discrete cytoplasmic foci known as stress granules (SG) for transient storage. Several cell proteins, including T cell intracellular antigen-1 (TIA-1), TIA-1 related protein (TIAR), and Ras-Gap-SH3 domain-binding protein (G3BP) are involved in stress granule assembly. Phosphorylation of eukaryotic translation initiation factor 2α (eIF2α) by protein kinase R (PKR) and other kinases prevents the assembly of the active ternary preinitiation complex eIF2-GTP-tRNA^{Met} and inhibits translation initiation and polysome assembly. TIA-1 and TIAR bind to these inactive translation initiation complexes as well as to the mRNA poly(A)⁺ and self-aggregate promoting the assembly of stress granules. Processing bodies (PB) another type of foci identified in the cytoplasm of eukaryotic cells, as defined herein, contain components of the 5′-3′ mRNA degradation pathway as well as the microRNA (miRNA)-dependent silencing protein, GW182. Processing bodies do not require eIF2α phosphorylation for their assembly. Although stress granules and processing bodies differ in their size and shape as well as in their mechanism of assembly, the two types of foci are found side by side in mammalian cells and a physical association between them was previously demonstrated by real-time fluorescence imaging. This study also showed that processing bodies are highly motile whereas the positions of stress granules are relatively fixed and that stress granules deliver mRNAs to associated processing bodies for degradation.

TIA-1 and TIAR are essential, multifunctional, nucleocytoplasmic shuttling proteins that are expressed in most types of cells and tissues. TIA-1 and TIAR contain three N-terminal RNA recognition motifs (RRM) and a glutamine-rich C-terminal domain, called prion related domain (PRD), that shares structural and functional characteristics with the aggregation domains of mammalian and yeast prion proteins. A recombinant TIA-1 protein lacking the three RNA binding domains was unable to bind poly(A)⁺ RNA and recruit it to stress granules. Deletion of the TIA-1 PRD inhibited protein aggregation and stress granule assembly. PRD aggregation is regulated by the molecular chaperone heat shock protein 70 (HSP70) and over expression of HSP70 prevented cytosolic aggregation of PRD. TIA-1 and TIAR also regulate translational silencing of a subset of cellular mRNAs via binding to AU-rich sequences in the 3′ noncoding regions (NCRs) of these mRNAs. In addition, TIA-1 and TIAR proteins interact specifically with the 3′-terminal stem loop structure of the West Nile virus minus-strand RNA [WNV3′(−)SL].

Though not wishing to be bound by any particular theory, it is believed that TIAR and TIA-1 may have several roles in viral replication, one role is in enhancing WNV replication by acting as transcription factors for plus strand synthesis. Another role involves stress granule formation. TIAR and TIA-1 form compartments much like stress granules that sequester viral plus strand RNA synthesis, so that translation of nascent plus strands into protein is inhibited. Another function is the inhibition of formation of stress granules by the cell in response to infection by a virus, which would prevent the shut off of cell protein translation and lead to a delay in the onset of apoptosis in the cells. Interference with the activity of TIAR and TIA-1 by the compositions of the present invention results in decreased synthesis of genome RNA, results in the shut off of cell protein synthesis and leads to the onset of apoptosis. The ability to affect the onset of apoptosis or to delay apoptosis can be applied to many disease states and treatments of such diseases by providing compositions that are effective in delaying or initiating apoptosis are contemplated by the present invention.

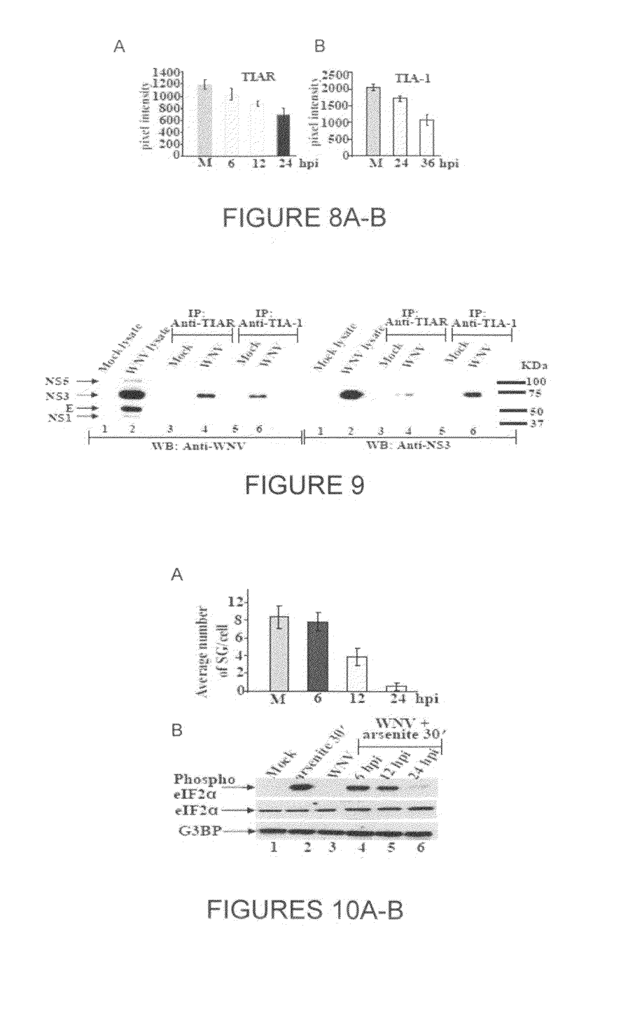

In normal cells that are not undergoing stress, TIA-1 and TIAR are distributed fairly evenly between the cytoplasm and nucleus. In response to stress, TIA-1 and TIAR aggregate with cell mRNAs in stress granules. The Examples herein show a unique distribution pattern for these proteins in cells infected with two different flaviviruses, WNV and DV. First TIAR and then TIA-1 relocated to the perinuclear region in flavivirus infected cells. In WNV infected cells, perinuclear foci of TIAR were detected by 6 hpi and a significant amount of TIAR was observed in this region by 12 hpi. With TIA-1, perinuclear foci were not detected until 24 hpi, and significant concentrations of TIA-1 in this region were not seen until 36 hpi. Interestingly, the initiation of TIA-1 relocation to the perinuclear region in infected cells did not begin until the majority of TIAR had already relocated to this region (See FIG. 8).

By 6 hpi, newly translated viral proteins were detectable in the cytoplasm by immunofluorescence, and by 12 hpi, the amount of viral protein had increased and was concentrated in the perinuclear region. Colocalization of bright foci of TIAR and of viral proteins was detected by 6 hpi, and the amount of colocalization progressively increased thereafter. These bright perinuclear foci also contained dsRNA identifying them as viral replication complexes. In vitro RNA-protein binding assays showed that TIAR bound 10 times more efficiently than TIA-1 to the WNV 3′ (−) SL RNA. The different kinetics of TIA-1 and TIAR redistribution in flavivirus infected cells may be attributable to the more efficient interaction between cytoplasmic TIAR and the 3′ ends of viral minus strand RNAs.

Though not wishing to be bound by any particular theory, the 3′ (−) SL is thought to contain promoter elements for genomic RNA initiation, and TIAR may function as a transcription factor for genomic RNA initiation. In fact, mutagenesis of the mapped protein binding sites on the WNV 3′ (−) SL RNA in a WNV infectious clone negatively affected genomic RNA synthesis. The kinetics of flavivirus RNA synthesis demonstrate an initial low peak of genomic RNA synthesis between 6 and 10-12 hpi followed by a switch to exponentially increasing genomic RNA synthesis. The level of genomic RNA synthesis infected cells correlates temporally with the extent of TIAR relocation to the perinuclear region.

The yield of WNV produced by TIAR−/− MEFs, which express three times more TIA-1 than wildtype MEFs, was reduced by only 6-to-8 fold as compared to TIA-1−/− or control MEFs. TIA-1 may function as a viral transcription factor when TIAR is not present. Indeed, colocalization of TIA-1 and WNV proteins in infected TIAR−/− MEFs occurred in a time course similar to that observed with TIAR in cells expressing both proteins. This data suggests that the ability of TIA-1 to relocate to the perinuclear region is not being actively prevented in infected cells. However, the TIA-1 may interact with a cell protein, which may make TIA unavailable for interaction with viral components until later times after infection.

Compositions contemplated by the present invention include compounds capable of inhibiting viral replication, by inhibiting the activity of host cell proteins involved in viral replication. Such compounds are capable of inhibiting the activity of host cell proteins in vitro and in vivo and show antiviral activity both in vitro and in vivo. The compositions of the present invention are capable of inhibiting the activity of host cell proteins without detrimentally affecting cellular viability.

One aspect of the present invention comprises administration of compositions comprising compounds such as nucleic acid constructs. For example, a nucleic acid construct can be a DNA molecule that is transcribed by the host cells to form decoy RNA molecules. The decoy RNA molecules then compete with the binding of host proteins, for example TIAR and TIA-1, to viral 3′ RNA. Other compounds include RNA molecules, small organic molecules, antibodies, peptides, peptoid, or polynucleotides that interfere with the binding of host proteins, for example TIAR and TIA-1, to the viral 3′ RNA. An alternative method of administration comprises administration of compositions of decoy RNA made in vitro. Routes of administration comprise those known in the art, though such compositions may be delivered, for example but not limited to, via injection or intranasal methods. The decoy RNA may comprise modified ribonucleic acid nucleosides that provide stability and resistance to nucleases. For example, 2-O-methyl RNA is very stable and is readily taken up by cells and is used for clinical applications. The present invention contemplates this and other modified RNAs.

Compositions for inhibiting the activity of host cell proteins such as p50, p60, p108, p105, p 52, p84, TIAR or TIA-1 can be provided as pharmaceutically acceptable formulations using formulation methods known to those of ordinary skill in the art. These formulations can be administered by standard routes. In general, the compositions may be administered by the topical, transdermal, oral, rectal or parenteral (i.e., intravenous, subcutaneous or intramuscular) route. In addition, the compositions may be incorporated into biodegradable polymers allowing for sustained release of the compound, the polymers being implanted in the vicinity of where drug delivery is desired, for example, in infected tissues or provided to the organism for sustained release of the compound to the entire organism, for example, via gastrointestinal absorption.

The dosage of the compound will depend on the condition being treated and the extent of infection, the particular compound, and other clinical factors such as weight and condition of the human or animal and the route of administration of the compound. It is to be understood that the present invention has application for human, veterinary or plant use. For example, for administration to humans, a dosage of between approximately 0.1-75 mg/kg/day, a dosage of between approximately 10-50 mg/kg/day, a dosage of between approximately 10-30 mg/kg/day. Alternatively, nucleic acid constructs provided in methods of gene therapy may be provided in dosages of picogram to micrograms/kg/day, between approximately 0.001 μg/kg/day to 100 μg/kg/day. Depending on the route of administration, the compound administered and the toxicity of that compound, a preferable dosage would be one that would yield an adequate blood level or tissue fluid level in the human, animal, plant, or insect that would effectively inhibit replication of the virus.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

Formulations suitable for topical administration to the skin may be presented as ointments, creams, gels and pastes comprising the ingredient to be administered in a pharmaceutical acceptable carrier. A topical delivery system is a transdermal patch containing the ingredient to be administered.

Formulations suitable for nasal administration, wherein the carrier is a solid, include a coarse powder having an appropriate particle size, microns, which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

The compositions of the present invention are used in methods of treatment of viral diseases, such as flavivirus infection. Such methods comprise administration of a composition effective in interfering or inhibiting viral replication in an individual having a flavivirus infection. Such compositions include compounds that are effective in interfering or inhibiting the interactions of host proteins in the viral replication pathway. In particular, such compositions interfere with the interactions by one or more of p50, p60, p108, p105, p52, p84, TIAR, or TIA-1 with viral nucleotides or alter other functions of the proteins, such as stress granule formation or apoptosis. An embodiment of a composition comprises a nucleic acid construct that mimics a portion of a flavivirus nucleic acid, such that the nucleic acid construct competes for the binding of one or more host cell proteins, such as p50, p60, p108, p105, p52, p84, TIAR, or TIA-1. An embodiment of a method of treatment of a flavivirus disease comprises administering to a human, animal, plant or insect an effective amount of a composition comprising a nucleic acid construct capable of competing for binding of one or more host cell proteins related to flavivirus replication, in an amount effective to inhibit or interfere with viral replication. An embodiment of a method of treatment of West Nile virus infection comprises administering to an individual, including a human or an animal, infected with West Nile virus, a composition comprising a nucleic acid construct capable of affecting one or more host cell proteins involved in West Nile virus replication, in an amount effective to interfere with or inhibit viral replication. An embodiment of a method of treatment of Dengue virus infection comprises administering to an individual, including a human or an animal, infected with Dengue virus, a composition comprising a nucleic acid construct capable of affecting one or more host cell proteins involved in Dengue virus replication, in an amount effective to interfere with or inhibit viral replication.

An embodiment of a composition comprises a compound that mimics a portion of a flavivirus nucleic acid, such that the compound competes for the binding of one or more host cell proteins, such as p50, p60, p108, p105, p52, p84, TIAR or TIA-1. An embodiment of a method of treatment of a flavivirus disease comprises administering to a human, animal, plant or insect an effective amount of a composition comprising a compound capable of competing for binding of one or more host cell proteins related to flavivirus replication, in an amount effective to inhibit or interfere with viral replication. An embodiment of a method of treatment of West Nile virus infection comprises administering to an individual, including a human or an animal, infected with West Nile virus, a composition comprising a compound capable of affecting one or more host cell proteins involved in West Nile virus replication, in an amount effective to interfere with or inhibit viral replication. An embodiment of a method of treatment of Dengue virus infection comprises administering to an individual, including a human or an animal, infected with Dengue virus, a composition comprising a compound capable of affecting one or more host cell proteins involved in Dengue virus replication, in an amount effective to interfere with or inhibit viral replication.

An aspect of the present invention comprises compositions that are effective in inhibiting or interfering with the interactions of host cell proteins, particularly TIA-1 and TIAR, that are involved in viral replication of flaviviruses. An aspect of the present invention further comprises compositions that are effective in inhibiting or interfering with the interactions of host cell proteins, particularly TIA-1 and TIAR, that are involved in viral replication of flaviviruses and particularly with the interactions of proteins with the viral proteins of flaviviruses. An aspect of the present invention comprises compositions that are effective in inhibiting or interfering with the interactions of TIA-1 and TIAR that are involved in viral replication of flaviviruses and particularly with the interactions of these proteins with the viral structural proteins (i.e., capsid (C), membrane (M), envelope (E)) and/or the viral non-structural proteins (i.e., NS1, NS2a, NS2b, NS3, NS4a, NS4b, and NS5). An aspect of the present invention comprises compositions that are effective in inhibiting or interfering with the interactions of TIA-1 and TIAR that are involved in viral replication of flaviviruses and particularly with the interactions of these proteins with viral non-structural proteins comprising NS3, NS2a, NS2b, NS4a and NS4b. For example, TIA-1 and TIAR are associated directly or indirectly with NS3. Methods of assaying for such compositions comprise adding compositions to uninfected cells, cells infected with a flavivirus (i.e., West Nile virus or Dengue virus), or cell-free systems, and measuring the reduction in viral replication, compared to infected cells or cell-free systems without the composition. An embodiment of the invention comprises adding a composition comprising a peptide that mimics a region of the flavivirus viral protein that interacts with TIA-1 and TIAR in an amount effective to inhibit or interfere with viral replication. An embodiment of the present invention comprises adding a composition comprising a peptide that mimics a region of the TIA-1 or TIAR protein that interacts with a flavivirus viral protein in an amount effective to inhibit or interfere with viral replication. An embodiment of the present invention comprises adding a composition comprising a compound that interferes with the interaction or bonding between flavivirus viral proteins and host cellular proteins, for example but not limited to TIA-1 or TIAR, in an amount effective to inhibit or interfere with viral replication.

An aspect of the present invention comprises compositions that are effective in inhibiting or interfering with the interactions of host cell proteins, including but not limited to TIAR and TIA-1, that are involved in flavivirus replication and particularly with the interactions of these proteins with the viral proteins of flaviviruses, including but not limited to Dengue virus and West Nile virus. Methods of assaying for such compositions comprise adding compositions to cells infected with DV or WNV, or cell-free systems for DV or WNV, and measuring the reduction in viral replication, compared to infected cells or cell-free systems without the composition. An embodiment of the invention comprises adding a composition comprising a peptide that mimics a region of a DV or WNV viral protein that interacts with TIA-1 and TIAR in an amount effective to inhibit or interfere with viral replication. One embodiment of the present invention comprises adding a composition comprising a peptide that mimics a region of a WNV viral protein NS3 that interacts with TIA-1 and TIAR in an amount effective to inhibit or interfere with viral replication. One embodiment of the present invention comprises adding a composition comprising a peptide that mimics a region of a host cellular protein, for example but not limited to TIA-1 or TIAR, that interacts with a WNV viral protein, such as NS3, in an amount effective to inhibit or interfere with viral replication. One embodiment of the present invention comprises adding a composition comprising a compound that interferes with the interaction or binding of a host cellular protein, for example but not limited to TIA-1 or TIAR, and a WNV viral protein, such as NS3, in an amount effective to inhibit or interfere with viral replication.

TIAR and TIA-1 may interact with a viral nonstructural protein as well as with the 3′ (−) SL RNA. WNV NS3 can be co-immunoprecipitated by both anti-TIA-1 and anti-TIAR antibodies. The four hydrophobic nonstructural proteins (NS2a, NS2b, NS4a, and NS4b) are tightly complexed with NS3 in dense membrane fractions, whereas the RdRp NS5 is more loosely associated with NS3. TIAR and TIA-1 may interact with the four hydrophobic nonstructural proteins, NS2a, NS2b, NS4a, and NS4b.

Flaviviruses initiate RNA synthesis de novo. The interaction of TIA-1 or TIAR with a protein in a flavivirus RdRp complex may enhance the preferential recognition of minus strand templates by these complexes. TIA-1 and TIAR do not bind to the 3′ terminal SL RNA of the genomic (+) RNA template. As the number of RdRp complexes progressively accumulates in infected cells, the synthesis of genomic RNA would be significantly and preferentially amplified over minus strand RNA. The binding of TIA-1 and TIAR to the 3′ (−) SL RNA may stabilize it allowing other necessary factors to assemble and initiation of genomic RNA synthesis to begin.

An aspect of a method of the present invention comprises inhibiting replication of a flavivirus, comprising, administering a composition capable of inhibiting the binding or interaction of at least one host cell protein involved for replication of a flavivirus, wherein the virus is a flavivirus, wherein the flavivirus is West Nile virus or Dengue virus. Methods include inhibition of the binding or association of at least one host cell protein, wherein at least one host cell protein is TIAR or TIA-1. Compositions used in such methods include compositions comprising proteins, polypeptides, and peptides, and include amino acid sequences that mimic one or more regions or domains of a flavivirus protein that interact with a host cell protein or that mimics one or more regions or domains of a host cell protein that interacts with a flavivirus protein. For example, a host cell protein is TIAR or TIA-1. For example, the flaviviruses may be WNV or DV. The compositions may comprise a small molecule, antibody, peptide, peptoid, or polynucleotide.

Methods of the present invention also comprise methods for treating a viral infection, comprising, administering to a human or animal having a viral infection, a composition that alters the interaction of one or more host cell proteins with a viral protein in an amount effective to inhibit or interfere with viral replication. The compositions comprise those taught herein, including but not limited to, a nucleic acid construct, a small molecule, an antibody, a peptide, peptoid or polynucleotide. The host cell proteins affected include but are not limited to, TIAR, TIA-1, p52, p84, p105, p108, p42, p50, or p60. Treatments include treatment of flaviviruses, for example, but not limited to, West Nile virus and Dengue virus.

Compositions contemplated by the present invention include compounds capable of inhibiting viral replication, by inhibiting the activity of host cell proteins involved in viral replication. Such compounds are capable of inhibiting the activity of host cell proteins in vitro and in vivo and show antiviral activity both in vitro and in vivo. In addition, the compositions of the present invention are capable of inhibiting the activity of host cell proteins without detrimentally affecting cellular viability.

One aspect of the present invention comprises administration of compositions comprising compounds, such as a peptide. For example, a peptide may comprise a DNA molecule, which encodes a peptide sequence, that is transcribed by host cells to form decoy peptides. The decoy peptides then compete with the binding of host proteins, for example TIAR and TIA-1, to flavivirus proteins. Other compounds include small organic molecules, antibodies, peptoid, polypeptides, or proteins that interfere with the binding of host proteins, for example TIAR and TIA-1, to the flavivirus proteins, for example but not limited to NS3. An alternative method of administration comprises administration of compositions of decoy peptides synthesized in vitro. Routes of administration comprise those known in the art, though such compositions are delivered, for example but not limited to, via injection or intranasal methods.

The compositions of the present invention are used in methods of treatment of flavivirus diseases. Such methods comprise administration of a composition effective in interfering or inhibiting flavivirus replication in an individual having a flavivirus infection. Such compositions include compounds that are effective in interfering or inhibiting the interactions of host proteins in the viral replication pathway. In particular, such compositions interfere with the interactions by one or more of p50, p60, p108, p105, p52, p84, TIAR or TIA-1 with viral nucleotides or alter other functions of the proteins, such as stress granule formation or apoptosis. An embodiment of a composition comprises a peptide that mimics a portion of a viral protein sequence that interacts with a host cell protein, such that the peptide competes for the binding of one or more host cell proteins, such as TIAR and TIA-1 among others. An embodiment of a method of treatment of a viral disease comprises administering to a human, animal, plant, or insect a composition comprising a peptide capable of competing for binding of one or more host cell proteins related to viral replication, in an amount effective to inhibit viral replication. An embodiment of a method of treatment of a flavivirus infection, for example but not limited to West Nile virus or Dengue virus, comprises administering to an individual, including a human or an animal, infected with a flavivirus, a composition comprising a compound capable of affecting one or more host cell proteins involved in flavivirus replications (i.e., West Nile virus replication or Dengue virus replication) in an amount effective to interfere with or inhibit viral replication.

An aspect of the present invention comprises compositions that are effective in inhibiting or interfering with the replication of flaviviruses by regulating cell-signaling cascades. An aspect of the present invention comprises compositions that are effective in inhibiting or interfering with the replication of flaviviruses by inducing the phosphorylation of eIF2α. Another aspect of the present invention comprises compositions that are effective in inhibiting or interfering with the replication of flaviviruses by activating the double-stranded RNA-activated serine/threonine protein kinase (PKR). Yet another aspect of the present invention comprises compositions that are effective in inhibiting or interfering with the replication of flaviviruses by suppressing the induction or expression of a PKR inhibitor or PKR-like ER kinase (PERK) inhibitor. Methods of assaying for such compositions may comprise adding compositions to cells infected with a flavivirus, or cell-free systems, and measuring the reduction in viral replication or the increase in stress granule formation compared to infected cells or cell-free systems without the composition or alteration in the phosphorylation patterns of proteins.

Flaviviruses are unique among animal positive strand RNA viruses in preventing rather than facilitating the shutoff of host cell translation. Flaviviruses replicate at significantly slower rates than most other positive strand RNA viruses in mammalian and avian cells. Rapid shutoff of cell protein synthesis with the resulting negative effect on cell viability would have a deleterious effect on flavivirus yields. Flavivirus infections do not induce stress granule formation, and cells infected with flaviviruses become increasingly resistant to the induction of stress granules by arsenite treatment with time after infection.

Flavivirus infections actively inhibit stress formation. The time course of the resistance to stress granule formation coincides with that of the relocation of TIAR to the perinuclear region rather than with the relocation of TIA-1 or with decreased phosphorylation of eIF2α. The sequestration of TIAR via its interaction with the viral 3′ (−) SL RNA and/or a viral nonstructural protein may be a reason for the observed decrease in stress granule formation in flavivirus infected cells. Although eIF2αwas phosphorylated in both TIA−/− and TIAR−/− MEFs, stress granule formation was impaired in TIA−/− MEFs but not in TIAR−/− MEFs, suggesting that TIAR alone cannot form stress granules. Considering that stress granule formation is inhibited to some extent when TIAR but not TIA-1 was sequestered in the perinuclear region, additional components necessary for stress granule formation may be sequestered in the perinuclear region of flavivirus infected cells. If self-aggregation of TIAR in the perinuclear region occurs, this may recruit additional stress granule components. Three cellular proteins, p50, p60, and p108 which bind to 3′ (−) SL RNA, may be components of stress granules. The assembly of pseudo-stress granule complexes around the viral minus strand RNA replication complexes may be a component of the remodeling of perinuclear membranes by virus infection to create an environment for efficient genomic RNA synthesis and encapsidation.

Complete resistance to stress granule induction and a significant decrease in eIF2αphosphorylation at S51 were not observed in WNV infected cells until 24 hpi, a time by which maximum levels of virus replication had been achieved. Unlike flaviviruses, both Semliki Forest virus (SFV), a positive-strand RNA alpha togavirus and some strains of reovirus, a double-stranded RNA virus, rapidly shut off host cell protein synthesis and induce stress granule formation via the induction of eIF2α (S51) phosphorylation. For both viruses, the phosphorylation of eIF2α, as well as the aggregation of TIA-1/TIAR into stress granules, were shown to be important for shut off of host cell protein translation. In cells subjected to environmental stresses, the activation of PKR and/or other cellular kinases leads to the phosphorylation of eIF2α. Activation of PKR followed by phosphorylation of eIF2α by PKR and the formation of stress granules was postulated to result in the shut off host cell translation in cells infected with SFV or Sindbis virus. The expression of P58^{IPK}, a PKR/PERK inhibitor, was shown to be decreased in L929 cells infected with host shutoff-inducing strains of reovirus and high levels of phospho-eIF2α were detected. In contrast, in cells infected with two flaviviruses, DV or Japanese encephalitis virus (MOI of 3), the expression of P581PK was significantly upregulated at 24 hpi. The timing of this upregulation was exactly coincident with the decrease in eIF2α phosphorylation observed in arsenite treated WNV infected (MOI of 5) cells. Yu et al. showed that the upregulation of P58^{IPK} was the result of activation of the cellular unfolded protein response by the accumulation of several different flavivirus proteins in infected cells. The initial shut off of stress granule formation in flavivirus infected cells may be due to the sequestration of TIAR other stress granule components in the perinuclear region of infected cells via interaction with viral components.

Processing bodies are involved in mRNA degradation and in translational repression. A similar decrease in processing body assembly was observed in both untreated and arsenite treated flavivirus infected cells. This decrease was temporally correlated with the initiation of TIA-1 relocation and the decrease in eIF2α phosphorylation in arsenite treated cells. The interference with processing body assembly may be related to activation of the unfolded protein response and may provide an additional mechanism to prevent translational repression and protect viral RNA and cellular mRNA from degradation.

An aspect of the present invention comprises compositions that are effective in inhibiting or interfering with the replication of flaviviruses by inducing the phosphorylation of eIF2α. Compositions capable of phosphorylating eIF2α may comprise PKR, PERK, heme-regulated initiation factor 2α kinase (HRI), general control non-derepressible-2 (GCN2), or other cell signaling proteins or molecules located upstream of eIF2α in its signaling cascade. Compositions capable of phosphorylating eIF2α further may comprise double-stranded RNA, interferons, viruses, replication-deficient viruses, small molecules, small organic molecules, cytokines, chemokines, viral vectors, proteins, peptides, peptide agonists, polypeptides, growth factors, nucleic acid, antibodies, peptoids, polyanions, or other cell signaling proteins, for example but not limited to PACT. For example, administration of a composition capable of phosphorylating eIF2α may comprise a DNA molecule, which encodes a peptide sequence or protein, that is transcribed and translated by host cells to form peptides or proteins capable of phosphorylating eIF2α. An alternative method of administration comprises administration of compositions synthesized in vitro. Routes of administration comprise those known in the art, though such compositions are delivered, for example but no limited to, via injection or intranasal methods.

An aspect of the present invention comprises compositions that are effective in inhibiting or interfering with the replication of flaviviruses by activating PKR. Compositions capable of activating PKR may comprise double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules, for example but not limited to PACT. For example, administration of a composition capable of phosphorylating eIF2α may comprise a DNA molecule, which encodes a peptide sequence or protein, that is transcribed and translated by host cells to form peptides or proteins capable of activating PKR. An alternative method of administration comprises administration of compositions synthesized in vitro. Routes of administration comprise those known in the art, though such compositions are delivered, for example but not limited to, via injection or intranasal methods.

Another aspect of the present invention comprises compositions that are effective in inhibiting or interfering with the replication of flaviviruses by suppressing the induction or expression of a PKR/PERK inhibitor. Compositions capable of suppressing the induction or expression of a PKR/PERK inhibitor may comprise small molecules, small organic molecules, cytokines, chemokines, proteins, peptides, nucleic acids, antibodies, peptoids, or other cell signaling proteins or molecules. For example, administration of a composition capable of suppressing the induction or expression of a PKR/PERK inhibitor may comprise a DNA molecule, which encodes a peptide sequence or protein, that is transcribed and translated by host cells to form peptides or proteins capable of suppressing the induction or expression of a PKR/PERK inhibitor. Alternatively, small interfering RNAs (siRNAs) may be used to reduce expression of a PKR/PERK inhibitor, for example but not limited to P58^{IPK}. An alternative method of administration comprises administration of compositions synthesized in vitro. Routes of administration comprise those known in the art, though such compositions are delivered, for example but limited to, via injection or intranasal methods.

An embodiment of the invention comprises adding a composition comprising a peptide, protein, nucleic acid, small molecule or the like capable of inducing the phosphorylation of eIF2α, activating PKR, or suppressing the induction or expression a PKR/PERK inhibitor in an amount effective to inhibit or interfere with viral replication.

An aspect of a method of the present invention comprises inhibiting replication of a virus, comprising, administering a composition capable of altering the cell signaling cascade of an infected cell, wherein the virus is a flavivirus, wherein the flavivirus is West Nile virus or Dengue virus. Methods include induction of the phosphorylation of eIF2α, activation of PKR, or suppression of the induction or expression a PKR/PERK inhibitor. Compositions used in such methods include composition comprising double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules.

Methods of the present invention also comprise methods for treating a viral infection, comprising, administering to a human or animal having a viral infection, a composition that alters cellular signaling cascades in an amount effective to inhibit or interfere with viral replication. The compositions comprise those taught herein, including but not limited to, double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules. The host cell proteins affected include but are not limited to, eIF2α, PKR, and PKR/PERK inhibitors (i.e., P58^{IPK}). Methods include treatment of flaviviruses, including but not limited to West Nile virus and Dengue virus.

Compositions contemplated by the present invention include compounds capable of inhibiting viral replication, by altering cellular signaling cascades involved in viral replication. Such compounds may be capable of altering cellular signaling cascades in vitro and in vivo. Such compounds may show antiviral activity both in vitro and in vivo.

The compositions of the present invention are used in methods of treatment of viral diseases. Such methods comprise administration of a composition effective in altering cellular signaling cascades and interfering with or inhibiting viral replication in an individual with a viral infection. Such compositions include compounds that are effective in altering cellular signaling cascades in the viral replication pathway. In particular, such compositions induce the phosphorylation of eIF2α, activate PKR, or suppress the induction or expression a PKR/PERK inhibitor. An embodiment of a composition comprises double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules, such that the composition induces the phosphorylation of eIF2α, activates PKR, or suppresses the induction or expression a PKR/PERK inhibitor. An embodiment of a method of treatment of a viral disease comprises administering to a human, animal or plant a composition comprising a double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules capable of inducing the phosphorylation of eIF2α, activating PKR, or suppressing the induction or expression a PKR/PERK inhibitor, in an amount effective to inhibit viral replication. An embodiment of a method of treatment of flavivirus infection (i.e., West Nile virus or Dengue Virus) comprises administering to an individual, including a human or an animal, infected with a flavivirus, a composition comprising a compound capable of affecting one or more cellular signaling cascades involved in flavivirus replication, such as but not limited to West Nile virus or Dengue virus, in an amount effective to interfere with or inhibit viral replication.

As used herein, individual can mean humans, animals, birds, insects, plants or other organisms that can be infected by viruses.

It must be understood that, as used in this specification and the appended claims, the singular forms “a” or “an” and “the” include plural referents unless the context clearly indicates otherwise.

All patents, patent applications, and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in the Examples and appended claims.

The present invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Materials and Methods Used Herein

CELLS Baby hamster kidney (BHK-21/WI2) cells (referred to hereafter as BHK cells) were used to prepare S100 cytoplasmic extracts or ribosomal salt wash cell extracts. BHK, CV-1 and Vero cells were maintained at 37° C. in a CO₂ incubator in Minimal Essential Medium (MEM) supplemented with 10 μg/ml gentamycin and 5% or 10% fetal calf serum (FCS).

TIAR-knockout C57BL/6 mice and TIA-1-knockout Balb/C mice were prepared as described previously. Embryo fibroblast cell lines were established from wild type (W4 and TIA^{+/+}43), TIAR-knockout (NaR4 and TIAR^{-/-}43) and TIA-1-knockout (a^{-/-}43 and TIA^{-/-}44) mouse embryos using the standard NIH 3T3 protocol.

To prepare control-reconstituted and TIAR-reconstituted (TIAR-REC) cell lines, TIAR-knockout (TIAR^{-/-}43) cells were transfected with a pSR-α-hygromycin vector containing full-length human TIAR cDNA (a gift from Dr. M. Streuli, Dana Farber Cancer Institute, Boston, Mass.) by the calcium phosphate method. Stable cell lines were established from clones that grew in the presence of hygromycin. Reconstituted cells were re-selected by growth in hygromycin for one week prior to use in experiments. These cell lines were maintained in MEM supplemented with 10% FCS, 10 mM HEPES and 10 μg/ml gentamycin in a CO₂ atmosphere at 37° C.

C57/BL/6 and C57/BL/6 TIAR−/− MEF cell lines were provided by Paul Anderson (Brigham and Woman's Hospital, Boston, Mass.) and grown in MEM supplemented with 10% FCS, 1×MEM non-essential amino acids (Invitrogen), 1 mM sodium pyruvate (Invitrogen), 2 mM glutamine (Invitrogen), and 10 μg/ml of gentamycin.

VIRUSES. Stocks of WNV, strain EG101, (titer=2×10⁸ PFU/ml) and Sindbis virus, strain SAAR 339, (titer=7×10⁹ PFU/ml) were prepared as 10% (w/v) newborn mouse brain homogenates. A stock of vaccinia virus, Wyeth strain, was prepared as a CV-1 cell lysate (titer=1.2×10⁸ PFU/ml). A stock of herpes simplex virus [HSV-1; strain H129 (H1)], was prepared as a media pool in Vero cells (titer=1.6×10⁸ PFU/ml) and vesicular stomatitis virus (VSV), strain Indiana, was prepared as a media pool in L cells (titer=4.8×10⁷ PFU/ml).

For virus growth experiments, confluent monolayers of wild-type or knockout cells in T25 flasks were infected with WNV at a multiplicity of infection (MOI) of 1, and culture fluid samples (0.5 ml) were harvested at different times post-infection (p.i.). At each time point, 0.5 ml of fresh media was replaced to maintain a constant volume in the flask. Harvested WNV samples were titrated in duplicate on BHK cells by plaque assay. Monolayers of cells in T25 flasks were also infected at an MOI of 1 with Sindbis virus, vaccinia virus, herpes simplex virus (HSV)-1, or vesicular stomatitis virus (VSV). Virus yields at different times post-infection were determined by plaque assay. Sindbis was plagued on BHK cells, vaccinia on CV-1 cells, HSV-1 on Vero cells, and VSV on BHK cells.

DV, strain Bangkok was provided by Walter Brandt (Walter Reed Army Institute of Research, Washington, D.C.). The titer of a 10% W/V suckling mouse brain homogenate was 2×10⁶ PFU/ml. Cells were infected with WNV at a MOI of 5 or 0.1 and with DV at a MOI of 0.1. Virus was adsorbed to cells for 1 hr at room temperature, the inoculum was removed and the cells were washed three times with serum free medium. MEM containing 5% FCS was added and the infected cells were incubated at 37° C. in a CO₂ incubator. To induce stress, cells were treated at various times after infection with 0.5 mM sodium m-arsenite (Sigma-Aldrich St. Louis, Mo.) in MEM for 30 min.

ANTIBODIES. Goat polyclonal antibodies to the C-terminus of TIAR, the C-terminus of TIA-1, eIF2α Texas red-conjugated bovine anti-mouse, FITC-conjugated donkey anti-goat, FITC-conjugated chicken anti-rabbit, and HRP-conjugated anti-goat, anti-mouse and anti-chicken antibodies were purchased from Santa Cruz Biotechnology, Inc (Santa Cruz, Calif.). Rabbit polyclonal anti-phospho-eIF2α (serine 51) and anti-rabbit immunoglobulin G antibody conjugated with horseradish peroxidase (HRP) were from Cell signaling (Beverly, Mass.), chicken polyclonal anti-G3BP was from Abcam (Cambridge, Mass.), mouse monoclonal anti-dsRNA antibody was from English & Scientific Consulting (Szirak, Hungary), and mouse anti-WNV NS3 monoclonal antibody was from R&D Systems (Minneapolis, Minn.). Rabbit anti-human DCP1a serum was a gift from J. Lykke-Anderson (University of Colorado, Boulder, Colo.). Mouse anti-WNV hyperimmune serum was from Robert Tesh (University of Texas Medical Branch, Galveston, Tex.). Mouse anti-DV hyperimmune serum was provided by Walter Brandt (Walter Reed Army Institute of Research, Washington, D.C.).

IN VITRO TRANSCRIPTION OF ³²P-LABELED RNA PROBES AND UNLABELED RNA TRANSCRIPTS. Plasmid p75 nt(−)3′ was previously constructed by P.-Y. Shi. A PCR product, PCRT73′ (−)SL, that consisted of the 75 3′ terminal nts of the WNV minus-strand RNA with three extra C's at the 5′ end copied from the T7 promoter, was amplified from plasmid p75 nt(−)3′ DNA using a M13 reverse primer ([SEQ ID NO 1] 5′-CAGGAAACAGCTATGACCATG-3′), and a forward primer ([SEQ ID NO 1] 5′-AGTAGTTCGCCTGTGTGAGC-3′). The 3′ (−)SL RNA was transcribed from the amplified PCR DNA. The T7 polymerase used for in vitro RNA transcription was expressed from BL21 cells containing pAR1219 (kindly provided by Dr. F. W. Studier, Brookhaven National Laboratory) and purified as described by Davanloo et al.

The methods used for in vitro transcription and gel purification of the ³²P-labeled

RNA probes and unlabeled competitor RNAs were described previously. Large scale batches of unlabeled RNAs, needed for RNA affinity columns, were prepared by scaling up the in vitro transcription reaction to 1 ml and extending the reaction time to 4 h.

RNA-AFFINITY COLUMN. In vitro transcribed WNV 3′ (−)SL RNA was oxidized with periodate in the presence of NaOAc (pH 5) and then attached to an agarose adipic acid matrix as described by Blyn et al. The RNA-matrix (1 ml) was poured into a 10 ml column and then equilibrated with column binding buffer [5 mM HEPES (pH 7.5), 25 mM KCl, 2 mM MgCl₂, 0.1 mM EDTA, and 2 mM dithiothreitol].

BHK S100 cell extracts prepared as described previously were subjected to ammonium sulfate precipitation prior to passage through an RNA-affinity column. Ammonium sulfate was first added to a final concentration of 16%. Ammonium sulfate was then added to the supernatant obtained from the first precipitation to a final concentration of 45% and the resulting pellet was resuspended in storage buffer. The pellet fraction was preincubated with non-specific competitors polyIC (1 mg/ml) and heparin (500 (μg/ml) at 4° C. for 10 min, and then passed over the RNA-affinity column three to five times. The column was then washed several times with column binding buffer and once with the same buffer containing 0.2 M NaCl. The bound proteins were eluted with column binding buffer containing 1 or 2 M NaCl. The eluted fractions were subjected to buffer exchange in a Centricon-30 (Amicon). Aliquots of each fraction were analyzed for RNA binding activity by gel mobility shift assay. Proteins were detected by Gold blot staining (Integrated Separation Systems). The proteins in the eluted fractions were then separated by SDS-PAGE, visualized by Coomassie blue staining. Protein bands were excised and peptides were generated by trypsin digestion. The peptides were separated by HPLC and the sequences of selected peptides were determined by automated liquid chromatography-tandem mass spectrometry using a Finnigan MAT LCQ ion trap mass spectrometer as described previously.

RNA-PROTEIN INTERACTION ASSAYS. Gel mobility shift and UV-induced crosslinking assays were performed as described previously. Prior to use in these assays, RNA probes were denatured by incubation at 90° C. for 10 min followed by renaturation by slow cooling to 60° C. and incubation at 60° C. for ˜2 min. The probe was then kept on ice until use.

IMMUNOPRECIPITATION OF UV-CROSSLINKED PROTEINS. Proteins in S100 cytoplasmic extracts were first crosslinked to ³²P-labeled WNV 3′ (−)SL RNA as described above. The cross-linked proteins were then incubated for 2 h at 4° C. with 1 μg/ml of anti-TIAR antibody [6E3, murine mAb IgG2a; (1)] or anti-TIA-1 antibody [ML29, murine mAb IgG1] that had been preincubated with Sepharose A CL-4B beads (Pharmacia). The precipitated complexes were pelleted by centrifugation at 300×g, washed twice with dilution buffer [0.1% Triton X-100 and 0.5% nonfat dry milk in TSA buffer (0.01 M Tris-HCl, pH 8.0, 0.14 M NaCl, 0.025% NaN₃)], once with TSA buffer and once with 0.05 M Tris-HCl (pH 6.8). The immunoprecipitated complexes were then separated by 10% SDS-PAGE and visualized by autoradiography.

COIMMUNOPRECIPITATION ASSAYS. BHK cells (2×10⁶) were mock infected or infected with WNV at a MOI of 5. At 24 hpi, cell lysates were prepared using lysis buffer containing 50 mM sodium phosphate (pH 7.2), 150 mM Nacl, 1% NP40, and Complete, Mini, EDTA-free protease inhibitor cocktail (Roche). Cell lysates were incubated on ice for 30 min, sonicated, and then centrifuged at 2000×g for 5 min at 4° C. The supernatant (250 μl/˜100 μg total protein) was precleared with protein A/G-magnetic beads (New England Biolabs, Beverly, Mass.) for 1 hr at 4° C. with rotation. The clarified supernatants were incubated with 1 μg of anti-TIA-1 or anti-TIAR antibody at 4° C. for 1 hr with rotation, then beads were added and incubated for 1 hr. Beads were collected magnetically, washed seven times with 1 ml of lysis buffer, and proteins eluted by boiling for 5 min. Proteins were separated by 12% SDS-PAGE, transferred to nitrocellulose membranes, and analyzed by Western blotting using either mouse anti-WNV or anti-NS3 antibody.

IMMUNOBLOTTING. BHK cells from a confluent monolayer in a T75 flask were trypsinized, pelleted by centrifugation for 3 min at 150×g, and washed three times with 1× phosphate-buffered saline (PBS). The cell pellet was resuspended in ice-cold lysis buffer (1×PBS, 1% Nonidet P-40, 0.5% sodium deoxycholate, 0.1% SDS) containing freshly added protease inhibitors (1× Complete, Roche), and passed through a 21-gauge needle four times. Nuclei were removed from the cytoplasmic extracts by centrifugation at 10,000×g for 10 min at 4° C. The total protein concentration in the extracts was determined using a Dc protein assay kit (BioRad). Proteins in 20 μg of extract were separated by 10% SDS-PAGE and then electrophoretically transferred to a nitrocellulose membrane (0.45 micron pore size, BioRad). The membrane was blocked with Blotto A [10 mM Tris-HCl, pH 8.0, 150 mM NaCl (TBS), 5% non-fat dry milk, 0.05% Tween 20] for 1 hr at room temperature or overnight at 4° C., and probed first with an anti-protein primary antibody and then with a horseradish peroxidase (HRP)-conjugated secondary antibody diluted in Blotto A. The membrane was washed three times with 1×TTBS (1×TBS containing 0.05% Tween 20) and then once with 1×TBS prior to incubation with Chemiluminescence Reagent (Santa Cruz Biotech) and detection of the proteins by autoradiography.

Mouse anti-TIAR monoclonal antibody 6E3 was used at 0.8 μg/ml and goat anti-TIA-1 polyclonal antibody (Santa Cruz Biotech) was used at 0.5 μg/ml. HRP-conjugated goat anti-mouse IgG and donkey anti-goat IgG were used at 0.2 μg/ml (Santa Cruz Biotech). Primary antibodies were diluted in 1× Tris-buffered saline (TBS) containing 5% bovine serum albumin (1:1000 for anti-phospho-eIF2α, 1:500 for anti-eIF2α, 1:3000 for anti-G3BP, and 1:500 for anti-WNV and anti-WNV NS3 antibodies) and the secondary antibody was diluted 1:2000 in 1×TBS containing 5% non-fat dry milk (NFDM).

INDIRECT IMMUNOFLUORESCENCE. Cells were grown to about 50% confluency in the wells of a two-chamber Lab-Tek II slide (Nalge Nunc International) and infected with WNV at a MOI of 5. At various times after infection, the cells were fixed with 2% paraformaldehyde for 10 min at room temperature, permeabilized with ice-cold methanol for 10 min, stained with a 1:100 dilution of a hyperimmune mouse anti-WNV antibody (Walter Reed Army Institute of Research) for 1 h, and then washed three times with PBS. The cell nuclei were then stained with Hoechst Dye (33258) and FITC-goat anti-mouse IgG (Jackson ImmunoResearch Laboratories) for 1 h and washed three times with PBS. The coverslips were mounted in vinol mounting media and viewed with a Nikon Eclipse 800 microscope equipped with epifluorescence optics and appropriate filters for detection of FITC, Texas red or Hoechst dye.

DETECTION OF INTRACELLULAR AND CELLULAR VIRAL PROTEINS BY IMMUNOFLUORESCENCE. BHK, C57BL/6, and TIAR−/− cells (2×10⁴) were seeded onto 3 mm cover slips (Fisher Scientific, Pittsburgh, Pa.) and grown to about 50% confluency. At various times after infection, cells were fixed in 4% paraformaldehyde (Sigma-Aldrich) in PBS for 10 min at room temperature, permeabilized with 100% chilled methanol for 10 min at −20° C., washed three times in PBS and incubated with blocking buffer [PBS containing 5% heat inactivated horse serum (Invitrogen)] overnight at 4° C. Primary antibodies were diluted in blocking buffer (1:1000 for anti-TIAR, anti-TIA-1 and anti-Dcap1, 1:200 for anti-dsRNA, and 1:100 for anti-WNV, anti-DV and anti-WNV NS3 antibody) and incubated with cells at 37° C. for 1 hr. Cells were then washed 3×10 min with PBS, and incubated for 1 hr at 37° C. with Texas red-labeled and FITC-labeled secondary antibodies diluted 1:300 in blocking buffer containing 0.5 μg/ml Hoechst 33258 dye (Molecular Probes, Bedford, Mass.) to stain the nuclear DNA. Cover slips were mounted in Prolong Gold antifade reagent (Invitrogen) and the cells were viewed and photographed with a Zeiss Confocal Microscope LSM 510 (Zeiss, Germany) using a 100× oil immersion objective. The images compared for each experimental series were collected using the same camera settings and the images were analyzed using Zeiss software version 3.5.

### Example 2

### Purification of the WNV 3′ (−) SL RNA-Binding Proteins

Three RNA-protein complexes, RPC1, -2 and -3, were detected in gel mobility shift assays done with a ³²P-WNV 3′(−) SL RNA probe and BHK 5100 extracts. UV-induced crosslinking assays indicated that these complexes contained four cell proteins (p42, p50, p60, and p108) that bound specifically to the WNV 3′ (−) SL RNA. An RNA affinity column was used to purify these viral RNA binding proteins.

In a preliminary experiment, a BHK S100 cytoplasmic extract that was prepared from ten T150 flasks of cells was subjected to precipitation with different concentrations of ammonium sulfate. Supernatant and pellet fractions were analyzed for viral RNA binding activity by gel mobility shift and UV-induced crosslinking assays. Although a pellet was obtained after precipitation with 16% ammonium sulfate, none of the four cell proteins that bound to the viral 3′ RNA were present in this pellet in detectable amounts. After precipitation with 45% ammonium sulfate, there was good recovery of RPC2 and RPC3, but only a small amount of RPC1 were detected in the pellet fraction by gel mobility shift assay. UV-induced crosslinking assays indicated that p60, p50, p42, but only a small amount of p108, were present in the pellet fraction.

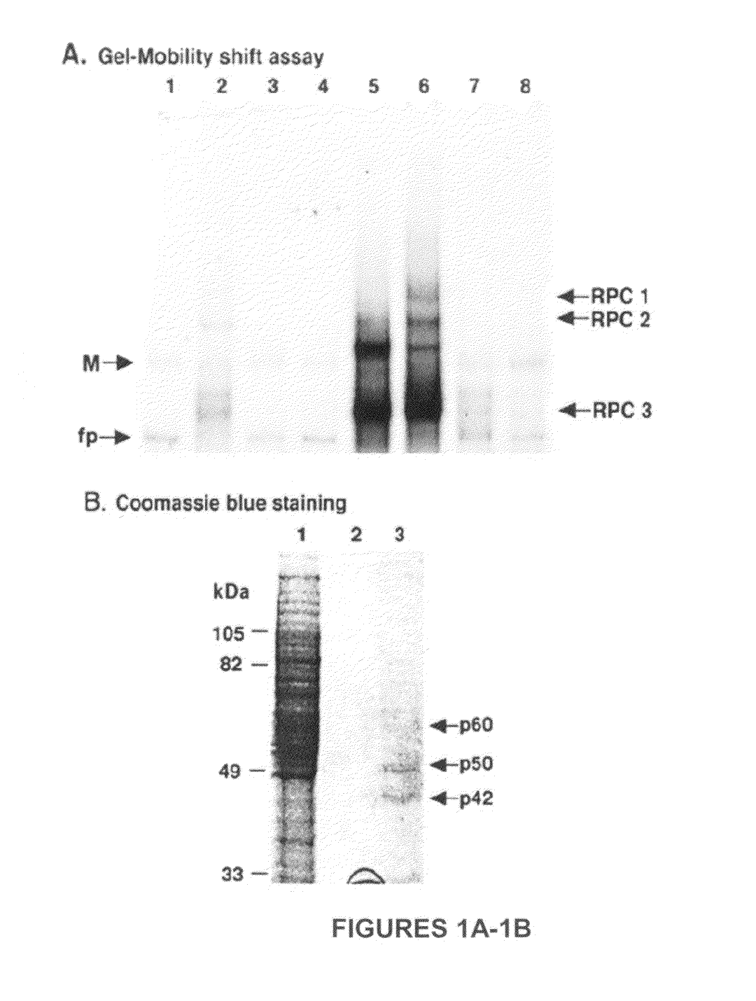

The proteins in the 45% ammonium sulfate pellet were resuspended in column binding buffer, incubated with non-specific RNA competitors, and then passed through a WNV 3′ (−) SL RNA affinity column several times. The column was washed and the bound proteins eluted. Each eluted fraction was concentrated with a Centricon-30 and assayed for viral RNA binding activity by gel-mobility shift and UV-induced cross-linking assays. FIG. 1 shows the analysis of WNV 3′(−) SL RNA-binding proteins in fractions eluted from an agarose-adipic acid hydrazide RNA affinity column. FIG. 1-A, Gel-shift assays, Lane 1, free probe; lane 2, final flow-through fraction from the RNA-affinity column; lane 3, first binding buffer wash fraction; lane 4, 0.2 M NaCl wash fraction; lanes 5 and 6, fractions eluted with 1 or 2 M NaCl respectively; lanes 7 and 8, fractions eluted from a “beads-only” control column with 1 or 2 M NaCl, respectively. For each of the fractions, 1 μl of a total of 100 μl was analyzed on the gel. The positions of the three RPCs are indicated by arrows. B. Coomassie blue staining of the eluted fractions from an agarose-adipic acid hydrazide RNA affinity column. Lane 1, aliquot of sample loaded on the affinity column (10 μl of 3 ml); lane 2, fraction eluted with 2 M NaCl from a “beads only” a control column (30 μl of 1000; lane 3, fraction eluted with 2 M NaCl from the RNA-affinity column (30 μl of 100 μl). The positions of the eluted proteins are indicated by arrows. The protein markers are shown on the left side of the gel. M, multimer of the probe; fp, free probe.

Little or no specific binding activity was detected in the flow-through fraction by gel-mobility shift assay (FIG. 1A, lane 2) or in the wash fractions (FIG. 1A, lanes 3 and 4). The majority of the RNA binding activity was eluted with 2 M NaCl (FIG. 1A, lane 6). Proteins in aliquot of the eluate was separated by 10% SDS-PAGE, transferred to a nitrocellulose membrane and stained with GoldBlot. Bands with molecular masses similar to those of three of the expected proteins (p60, p50 and p42), as well as some background bands were observed (data not shown). The p42 band was clearly the strongest band. The remainder of the eluted protein was then electrophoresed on one lane of a 10% SDS-PAG and then stained with Coomassie blue (FIG. 1B, lane 3). The p42 and p50 bands were excised from the gel and peptides were generated by trypsin digestion. The peptides were separated by HPLC and the sequences of selected peptides were determined by automated liquid chromatography-tandem mass spectrometry (Beckman Research Institute of the City of Hope). Insufficient unique sequence for p50 was obtained to allow the identification of this protein. The sequences of four peptides obtained from p42 were identical to sequences found in both TIA-1 and TIAR, while the sequences of two additional p42 peptides were unique to TIAR. TIAR and TIA-1 are closely related RNA binding proteins that bind U-rich sequences interspersed with As. Both proteins contain three N-terminal RRM domains, each approximately 100 amino acids in length, and a C-terminal auxiliary domain of approximately 90 amino acids. TIAR and TIA-1 share 80% overall amino acid identity, with the highest degree of similarity in RRM domain 3 (91% identity) and the lowest degree of similarity (about 50% identity) in the C-terminal auxiliary domain. The data suggest that p42 is TIAR. However, because of the high degree of sequence homology between TIAR and TIA-1, the possibility that TIA-1 also binds specifically to the WNV 3′ (−) SL RNA could not be ruled out.

Studies to identify the other three cell proteins that bind to the WNV 3′ (−) RNA are in progress. Previous preliminary studies showed that neither anti-EF-1α nor anti-La antibody produced a supershift when added to S100 cytoplasmic extracts incubated with the WNV 3′ (−) SL RNA probe.

### Example 3

### Confirmation that TIAR and TIA-1 Bind to the WNV 3′ (−) RNA

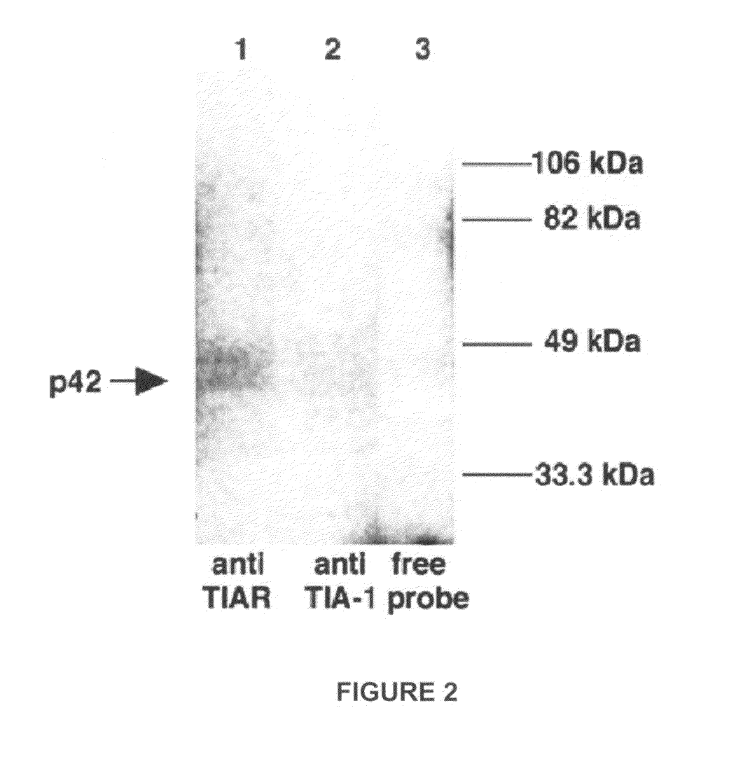

Anti-TIAR (6E3) and anti-TIA-1 (ML-29) antibodies were used to immunoprecipitate UV-induced crosslinked WNV 3′ (−) SL RNA-protein complexes from BHK S100 cytoplasmic extracts after treatment with RNase. Results from this experiments are shown in FIG. 2. BHK S100 cytoplasmic extracts from BHK cells were incubated with a ³²P-labeled WNV 3′(−) SL RNA probe. The complexes were cross-linked by exposure to UV light, treated with RNase and then precipitated with anti-TIAR or anti-TIA-1 antibody. The precipitates were analyzed by 10% SDS-PAGE. Lane 1, anti-TIAR antibody was added; lane 2, anti-TIA-1 antibody was added; lane 3, free probe. The expected UV-induced cross-linked p42 product is indicated by an arrow. The protein markers are shown on the right side of the gel. Even though four cell proteins (p108, p60, p50, and p42) that crosslinked to the viral RNA probe were present in these extracts, only the anti-TIAR antibody precipitated the p42-WNV 3′ (−) SL RNA complex (FIG. 2, lane 1). Anti-TIA-1 antibody also precipitated the p42-WNV 3′ (−) SL RNA complex (FIG. 2, lane 2). However, the band obtained after immunoprecipitation with anti-TIA-1 antibody was not as strong as that seen with the anti-TIAR antibody. These data suggest that the p42 band detected in UV crosslinking experiments with S100 extracts contained both TIAR and TIA-1.

### Example 4

### Analysis of the Specificities of the Viral RNA-Cell Protein Interactions

Purified GST-TIAR and GST TIA-1 fusion proteins (41) were tested for their ability to bind to the WNV 3′ (−) SL RNA in a gel mobility shift assay. The results of preliminary experiments showed that at least a four times higher concentration of GST-TIA-1 was required to detect binding in gel mobility shift assays as compared to GST-TIAR (data not shown). Therefore, different concentrations of GST-TIA-1 (200 nM) and GST-TIAR (50 nM) were used for the representative competition gel shift assays shown in FIGS. 3 A and B. Although the predominant gel shift band observed with both of the fusion proteins migrated to the middle of the gel, additional slower and faster migrating complexes were also observed. The slower migrating bands most likely contain aggregated complexes, since the density of these bands increased with increasing protein concentration (data not shown). The faster migrating bands most likely contained breakdown fragments that retained the RRM2 region containing the viral RNA binding site (see FIGS. 3, C and D).

FIG. 3 shows the analysis of the specificities of the interactions between the WNV 3′(−) SL RNA and the recombinant TIAR or TIA-1 proteins. FIG. 3-A. Competition gel-shift assays with a purified GST-TIA-1 fusion protein. Lane 1, free probe; lane 2, probe plus 200 nM of purified GST-TIA-1 fusion protein; lanes 3 to 14, probe plus 200 nM of purified GST-TIA-1 fusion protein and the indicated competitor RNA. FIG. 3-B. Competition gel shift assays with a purified GST-TIAR fusion protein. Lane 1, free probe; lane 2, probe plus 50 nM of purified GST-TIAR fusion protein; lanes 3 to 14, probe plus 50 nM of purified GST-TIAR fusion protein and the indicated competitor RNA. SC, specific competitor-unlabeled 75 nt WNV 3′(−) SL RNA; M, multimer of the probe; fp, free probe. FIG. 3-C. Purified GST-fusion proteins (500 nM), each containing a single RRM domain of TIA-1, were analyzed by gel mobility shift assay. Lane 1, free probe; lane 2, probe plus the GST-TIA-1 RRM1; lane 3, probe plus the GST-TIA-1 RRM 2; lane 4, probe plus the GST-TIA-1 RRM3. M, multimer of the probe; fp, free probe. FIG. 3-D. Purified GST-fusion proteins (200 nM), each containing a single RRM domain of TIAR, were analyzed by gel mobility shift assay. Lane 1, free probe; lane 2, probe plus the GST-TIAR RRM1; lane 3, probe plus the GST-TIAR RRM2; lane 4, probe plus the GST-TIAR RRM3.

Unlabeled WNV 3′ (−) SL RNA (50 or 100 ng) was used as the specific competitor and competed efficiently with the labeled probe (FIG. 3A, lanes 3 and 4; FIG. 3B, lanes 3 and 4). The nonspecific competitors, poly (IC), poly (A), poly (G), and poly (C), showed little or no competition even at concentrations of 500 ng or 1 μg (FIGS. 3, A and B). tRNA (100 or 500 ng) partially competed, but with a lower efficiency than the specific competitor (FIG. 3A, lanes 6 and 7 and FIG. 3B, lanes 6 and 7). As expected from previous studies showing that TIAR and TIA-1 bound to U rich sequences (1), poly U competed efficiently (FIG. 3A, lane 14 and FIG. 3B, lane 14). These data indicate that both the GST-TIAR and GST-TIA-1 proteins bind specifically to the WNV 3′ (−) SL RNA.

TIA-1 and TIAR each contain three RRM domains. To determine whether one of these RRM domains contains the major binding site for the WNV 3′ (−) SL RNA, purified truncated GST fusion proteins, GST-TIA-1 RRM1, GST-TIA-1 RRM2, and GST-TIA-1 RRM3 (FIG. 3C, lanes 2 to 4), and GST-TIAR RRM1, GST-TIAR RRM2, GST-TIAR RRM3 (FIG. 3D, lanes 2 to 4), were tested for their ability to bind to the WNV 3′ (−) SL RNA in gel mobility shift assays. Only the GST-TIA-1 RRM2 (FIG. 3C, lane 3) and the GST-TIAR RRM2 (FIG. 3D, lane 3) were able to bind the WNV 3′ (−) SL RNA. These data suggest that the major WNV 3′ (−) SL RNA binding site in both TIAR and TIA-1 is RRM 2.

### Example 5

### Determination of the Relative Dissociation Constants (K_{D}) for the Viral RNA-Cell Protein Interactions

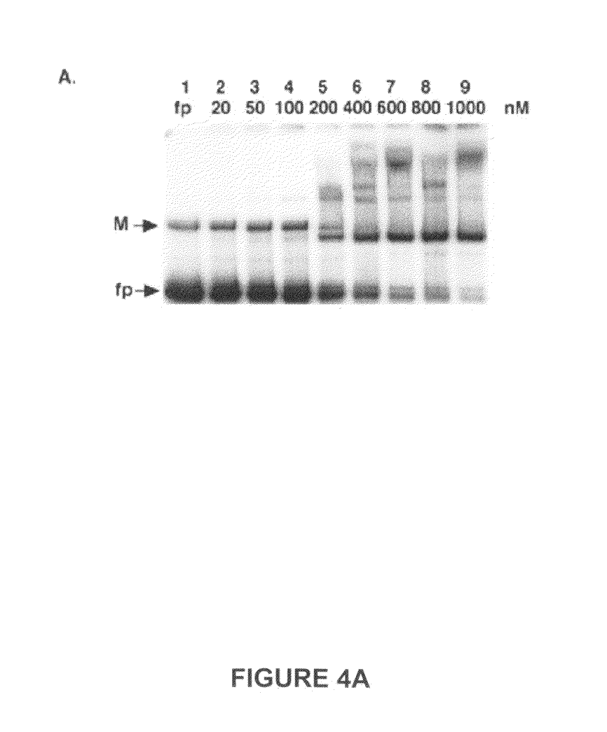

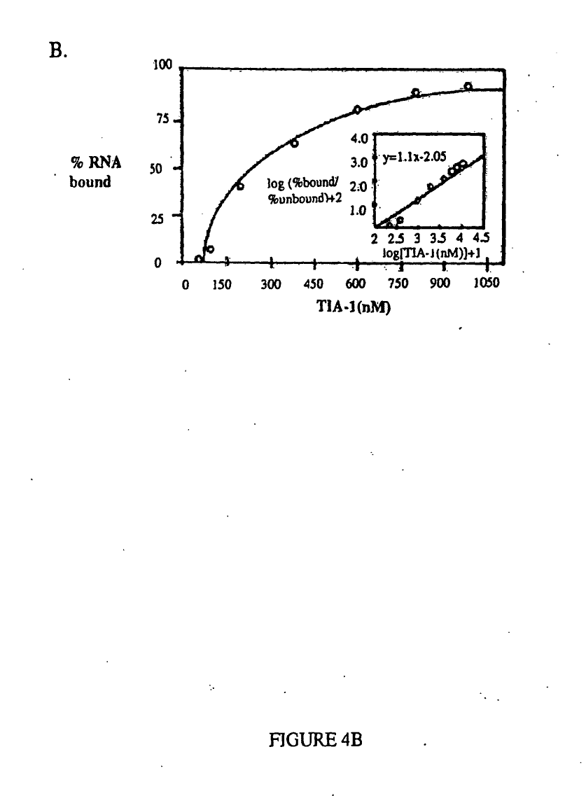

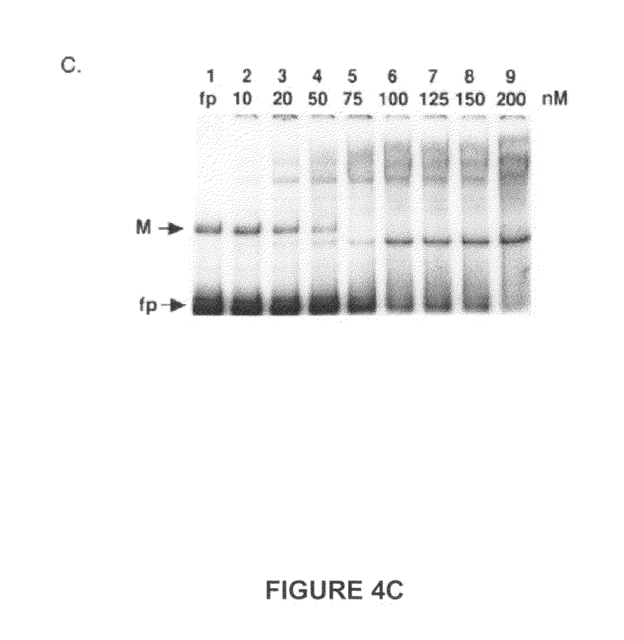

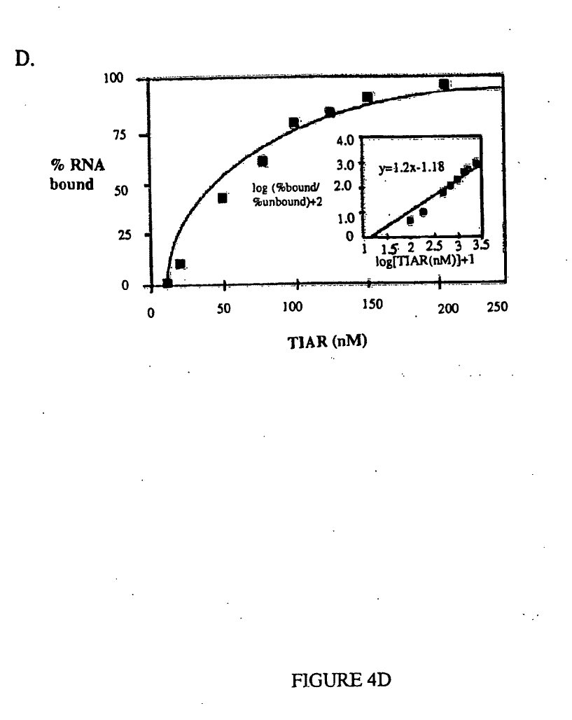

FIG. 4 shows quantification of the protein-RNA interactions. FIG. 4-A shows a representative gel mobility shift assay done with increasing amounts of the GST-TIA-1 RRM2 protein and a constant amount of WNV 3′(−) SL RNA. Lane 1, free probe; lanes 2 to 9, probe plus the GST-TIA-1 RRM2 in the amounts indicated. M, multimer of the probe; fp, free probe. FIG. 4-B. The percent ³²P-WNV 3′(−) SL RNA bound was plotted against the concentration of TIA-1 to generate a theoretical saturation binding curve. Inset, the data from the saturation binding curve were transformed as described previously. The stoichiometry of the interaction of TIA-1 with the WNV 3′(−) SL RNA, as determined by the slope of the line in the inset graph, was about 1:1. The dissociation constant was calculated using the equation log (% bound % unbound)+2=n{log [TIA-1 (nM)]+1}−logK_{d}. The Kd was estimated to be 112 nM for TIA-1. FIG. 4-C. A representative gel mobility shift assay done with increasing amounts of the GST-TIAR RRM2 protein and a constant amount of WNV 3′(−) SL RNA. Lane 1, free probe; lanes 2 to 9, probe plus the GST-TIAR RRM2 in the amounts indicated. FIG. 5D. The percent ³²P-WNV 3′(−) SL RNA bound was plotted against the concentration of TIAR to generate a theoretical saturation binding curve. Inset, the data from the saturation binding curve were transformed. The Kd was estimated to be 15 nM for TIAR. The stoichiometry of the interaction of TIAR with the WNV 3′(−) SL RNA, as determined by the slope of the line of the inset graph, was about 1:1. M, multiprobe; fp, free probe.

Gel mobility shift assays were performed using different amounts of GST-TIA-1 RRM2 or GST-TIAR RRM2 protein and a constant amount of the ³²P-WNV 3′ (−) SL RNA. Although gel shift bands were observed with 10 nM of GST-TIAR RRM2, bands for GST-TIA-1 RRM2 were first observed when 50 nM of protein were used (FIGS. 4A and 4C). A theoretical saturation binding curve was generated by plotting the percentage of bound WNV 3′ (−) SL RNA versus the concentration of either GST-TIA-1 RRM2 or GST-TIAR RRM2. The data from the saturation binding curve were transformed as described previously. The relative K_{d} for the interaction between GST-TIA-1 RRM2 and the WNV 3′ (−) SL RNA was estimated to be about 1.12×10⁻⁷ M (FIG. 4A), while the relative K_{d} for the interaction between GST-TIAR RRM2 and the WNV 3′ (−) SL RNA was estimated to be about 1.5×10⁻⁸ M (FIG. 4C). The slope (n) of the line represents the ratio of GST-TIA-1 RRM2 or GST-TIAR RRM2 molecules to WNV 3′ (−)SL RNA molecules in each RNA-protein complex (FIGS. 4B and D, insets). For both proteins, the slope was calculated to be about 1 (1.1 for TIA-1 and 1.2 for TIAR), suggesting that approximately one TIAR or TIA-1 molecule binds to each WNV 3′ (−) SL RNA molecule. Similar K_{d} and n values were obtained from four independent experiments with standard deviations of ±15 nM and ±0.15 for GST-TIA-1 RRM2 and ±5 nM and ±0.1 for GST-TIAR RRM2, respectively. These data indicate that the relative binding activity of the TIAR-RRM2 for the WNV 3′ (−) SL RNA is more than 10 times higher than that of the TIA-1-RRM2 for the same RNA. Although the RRM2 domain was shown to contain the main binding site for the viral RNA (FIG. 3), both proteins also contain two additional RRM domains that are likely to participate in stabilizing the RNA-protein interaction. The relative binding activities of the complete proteins for the viral 3′ RNA therefore would be expected to be somewhat higher.

### Example 6

### Effect of TIAR and TIA-1 on the Replication of WNV

As one means of assessing the effect of the TIAR and TIA-1 proteins on WNV replication, virus growth was compared in TIAR-knockout, TIAR-reconstituted, TIA-1-knockout, and control murine embryo fibroblast cell lines. Confluent cell monolayers were infected with WNV at an MOI of 1. Culture fluid samples were taken at 2, 8, 12, 24, 28, and 32 hr postinfection (p.i.). A representative growth curve of WNV in wild type (W4), TIAR-knockout (NaR4), and TIA-1-knockout (a^{-/-}43) cells is shown in FIG. 5A. Virus titers were expressed as PFU/cell, because the various cell lines grew to different but characteristic densities when confluent. The peak titer of WNV produced by TIAR-knockout cells was significantly lower (6-8 fold) than that produced by control cells (FIG. 5A). WNV grew to comparable peak titers in TIA-1-knockout cells and control cells, but peak virus levels were not attained until 6 hr later in TIA-1-knockout cells. Similar results were obtained with an additional set of separately derived control and knockout cell lines (data not shown) suggesting that the decrease in WNV replication observed in the knockout cells was not due to a peculiarity of a single cell line.

The efficiency of infection of these cells with WNV was investigated by indirect fluorescence. Control, TIAR-knockout and TIA-1 knockout cells were infected with WNV for 24 h, 28 h, or 32 h, fixed, and then stained with Hoechst dye and anti-WNV antibody. At 24 hr post infection, bright virus-specific perinuclear staining was observed in about 40% of the control and TIA-1-knockout cells. However, the stained perinuclear areas in the infected control cells were generally wider than those in the TIA-1-knockout cells. Although a similar percentage of TIAR-knockout cells showed virus-specific perinuclear staining at 24 hr, the fluorescence in these cells was faint and the areas of staining were focal. The intensity of the perinuclear staining in the infected TIAR-knockout cells increased somewhat by 28 h post infection and thin perinuclear rings were observed in some cells. At 32 h, although the intensity and distribution of the fluorescence had increased in the WNV-infected TIAR-knockout cells, only about 10-20% of the cells contained broad, brightly stained perinuclear rings. These results suggest that WNV infects similar numbers of cells in the three types of cultures but that virus replication is most efficient in the control cells, slightly less efficient in the TIA-1-knockout cells and least efficient in the TIAR-knockout cells.

### Example 7

### Growth of Other Types of Viruses in TIAR-Knockout and TIA-1-Knockout Cells

To determine whether other types of viruses also showed reduced growth in TIAR-knockout cells, control, TIAR-knockout, and TIA-1-knockout cells were infected with Sindbis virus, vaccinia virus, VSV or HSV-1 at an MOI of 1. Sindbis virus is another plus strand RNA virus but from the alpha togavirus family. VSV, a rhabdovirus, is a minus strand RNA virus, while vaccinia, a poxvirus, is a DNA virus. Similar to WNV, these three viruses replicate in the cytoplasm of infected cells. HSV-1, a herpes virus, is a DNA virus that replicates in the nucleus. Culture fluid samples were harvested at the indicated times after infection and titered by plaque assay. Representative growth curves obtained for each of the viruses are shown in FIG. 5C through F. Confluent monolayers of control (W4), TIA-1-knockout (a^{-/-}43) and TIAR-knockout (NaR4) cells were infected with (A) WNV, (C) VSV, (D) Sindbis virus, (E) HSV-1, or (F) vaccinia virus at an MOI 1. Confluent monolayers of control (W4), TIAR-reconstituted (TIAR-Rec) or control reconstituted (Cont-Rec) cells were infected with (B) WNV at MOI 1. Culture fluid samples were taken at the indicated hours post-infection (p.i.) and titered by plaque assay.

VSV (FIG. 5C) and Sindbis virus (FIG. 5D) grew to similar titers in TIAR-knockout and control cells, whereas in TIA-knockout cells, both of these viruses grew to significantly higher titers suggesting that the presence of TIA-1 had a negative effect on the growth of these viruses. HSV-1 also grew to significantly higher levels in TIA-1 knockout cells than in control cells (FIG. 6E). However, the growth of HSV-1 in TIAR-knockout cells was also more efficient than in control cells, but not as efficient as in TIA-knockout cells. The efficiency of growth of vaccinia virus (FIG. 5F) in all three types of cells was similar. Because the majority of the vaccinia progeny virus is cell associated, the extracellular virus titers detected were significantly lower than those for the other viruses. These results indicate that only the growth of WNV was less efficient in the TIAR-knockout cells.

To further investigate the effect of TIAR on viral growth, the growth of WNV in a TIAR-reconstituted stable cell line, TIAR-REC, was tested. Another stable cell line, Cont-REC, which had been transfected with the same vector, but did not express TIAR at detectable levels (FIG. 6E), was used as a control for possible nonspecific effects of the vector. FIG. 5B shows representative WNV growth curves obtained with control, TIAR-REC and Cont-REC cells. Although the peak titer of WNV produced by TIAR-reconstituted cells was higher than that produced by Cont-REC cells, it was lower than that produced by control cells.

### Example 8

### Comparison of the Relative Amounts of TIAR and TIA-1 Proteins in the Various Cell Lines

The relative amounts of TIAR and TIA-1 in cytoplasmic extracts from each of the cell lines were estimated by immunoblotting using protein-specific antibody. Previous studies showed that two isoforms generated by alternative splicing exist for both TIA-1 and TIAR. The two TIA-1 isoforms, 42 kDa TIA-1a and 40 kDa TIA-1b, differ from each other by an 11 amino acid deletion. These isoforms are usually found in cells in a 1:1 ratio. The two TIAR isoforms, 42 kDa TIARa and 40 kDa TIARb, differ from each other by a 17 amino acid deletion. Because TIARb is six times more abundant in cells than TIARa, it is the only isoform that is detected by Western blotting. Representative Western blots are shown (FIGS. 6A, B, D, and E). Twenty μg of total cell protein were run on each lane. The TIAR protein and two isoforms of the TIA-1 protein are indicated by arrows. Quantification of the relative amounts of protein in the various types of cells is shown in FIGS. 6 C and F. The relative amount of each protein in the control cells was defined as 1. The relative amounts of the proteins in other cell lines were expressed as the ratio of the protein band intensity divided by the density of the band in control cells. The values shown are means of the values obtained from 3 to 5 separate experiments.

As expected, no TIA-1 protein was detected in cytoplasmic extracts from TIA-1-knockout cells (FIG. 6A) and no TIAR protein was detected in cytoplasmic extracts from TIAR-knockout cells (FIG. 6B). The level of the TIAR protein in cytoplasmic extracts from TIA-1 knockout cells (a^{-/-}43) was slightly decreased (FIG. 6B), but the amount of TIA-1 protein in the cytoplasm of TIAR-knockout cells was significantly increased (by 3.3 fold) as compared to the amounts of these proteins present in the control (W4) cells (FIGS. 6A and C). These data indicate that the level of TIA-1 is down-regulated by TIAR. No significant differences in the cytoplasmic levels of either protein were observed after WNV infection (at 5 or 8 hr p.i.) in the various cell lines tested (data not shown).

The amount of TIAR protein detected in the TIAR-REC cells was about 80% of that detected in control (W4) cells (FIGS. 6E and F), while no TIAR protein was detected in Cont-REC cells (FIG. 6E). The amount of TIA-1 protein in the TIAR-REC cells was 2 fold higher, while the amount of TIA-1 protein in Cont-REC cells was 3.5 fold higher, as compared to control W4 cells (FIGS. 6D and F). These data indicate that the TIAR-REC cells had intermediate levels of the two proteins.

### Example 9

### Comparison of TIAR and TIA-1 cDNA Sequences from Cells Obtained from Flavivirus Resistant and Susceptible Mice

A single, dominant gene, Flv, that maps to chromosome 5 confers a flavivirus resistance phenotype in mice. Data from previous studies showed that resistant mice as well as cells obtained from a number of different tissues of resistant mice produced significantly lower titers of flaviviruses than did congenic susceptible mice or cells and that genomic RNA levels, but not minus strand viral RNA levels, were lower in resistant cells. Since both TIAR and TIA-1 bind to the WNV 3′(−)SL RNA and this SL is located at the site of initiation of genomic RNA synthesis, it was of interest to determine whether the sequences of TIAR and TIA-1 cDNAs differed in cells from resistant C3H/He and congenic susceptible C3H.RV mice. Cell RNA was extracted from resistant and susceptible embryo fibroblasts with TRIZOL-LS (Life/Gibco) according to the manufacturer's instructions. Using primers designed from mouse (strain 129 SVJ) TIA-1 and TIAR cDNA sequences previously reported by Beck et al. (1996), cDNAs were amplified by RT-PCR from cell mRNA and TA cloned into pCR 2.1-TOPO (Invitrogen). At least three cDNA clones for each isoform were sequenced. The sequences obtained for the two TIAR isoform cDNAs and for the two TIA-1 isoform cDNAs from resistant C3H.RV were identical to those of the comparable isoforms obtained from susceptible C3H/HE cells. These sequences were also identical to the previously reported sequences for these proteins from 129SVJ mice (Accession numbers: U55861 and U55862) by Beck et al. Also, as assessed by Western blotting, the expression levels of the TIAR and TIA-1 proteins in resistant cells and susceptible cells were similar. These data indicate that neither TIAR nor TIA-1 is the product of the Fly gene.

### Example 10

### TIA-1 and TIAR Colocalize with WNV and DV Proteins in Infected BHK Cells

A previous study showed that in C57/BL/6 TIAR−/− mouse embryo fibroblasts (MEFs), the replication of WNV was reduced by 6- to 8-fold compared to control MEFs, whereas WNV grew to the same levels in TIA-1−/− MEFs and control MEFs. This was consistent with the 10 fold higher binding efficiency of TIAR for the WNV3′(−)SL as compared to TIA-1. To determine whether a flavivirus infection altered the distribution of these cell proteins, BHK cells were infected with WNV at MOI of 5 and at 3, 6, 12, and 24 hpi, the cells were fixed, permeabilized, incubated with anti-TIA-1 or anti-TIAR and then anti-WNV antibody, and visualized by confocal microscopy. TIAR and TIA-1 were evenly distributed throughout mock infected cells. Under these infection conditions, WNV proteins were detected in about 90% of the cells. WNV proteins were faintly detected by 6 hpi. Although at 6 hpi, TIAR was still found in both the cytoplasm and nucleus, this protein was also concentrated in a few bright foci in the cytoplasm. Merged images obtained at 6 hpi showed colocalization of TIAR foci with foci of viral protein. Foci of TIA-1 were not observed at this time. By 12 hpi, the majority of TIAR was in the cytoplasm and the amount of WNV protein had increased and was concentrated in the perinuclear region. Again, colocalization of TIAR but not TIA-1 with the viral proteins was observed mainly in perinuclear foci. By 24 hpi, the intensity of WNV protein staining had increased significantly and TIAR strongly colocalized in the perinuclear region with the WNV proteins. In contrast, only a few bright cytoplasmic foci of TIA-1 colocalized with the WNV proteins by 24 hpi (a pattern similar to that seen at 6 hpi with TIAR). Strong colocalization of TIA-1 with the WNV proteins was observed by 36 hpi. Quantification of the amount of TIAR or TIA-1 in the nucleus confirmed that the distribution of both TIA-1 and TIAR was altered with time after WNV infection but the redistribution of TIAR occurred more rapidly (FIG. 8). Laser scanning confocal microscopy of mock infected or WNV infected (MOI of 5) BHK cells stained with anti-WNV and anti-TIAR antibody and BHK cells stained with anti-WNV and anti-TIA-1 antibody at the indicated times after WNV or mock infection at the indicated time post infection was performed. Quantification of the amount of TIAR (FIG. 8A) or TIA-1 (FIG. 8B) in the nucleus of mock-infected cells (M) and WNV-infected cells at the indicated times after infection was performed. The relative pixel intensity in the nuclei of 20 cells at each time after infection was measured and the mean values were plotted. Error bars indicate the standard deviation of the mean.

It was previously reported that the expression of TIA-1 was three times higher in TIAR−/− than in wildtype MEFs. To determine if the time course of TIA-1 redistribution was altered when TIAR was not present, TIAR−/− MEFs were infected with WNV (MOI of 5) and the locations of TIA-1 and WNV proteins were analyzed at 12 and 24 hpi. TIA-1 was equally distributed in both the cytoplasm and nucleus of mock infected TIAR−/− MEFs, but these cells were significantly larger than BHK cells. The time course of appearance, staining intensity, and distribution of the WNV proteins in these cells were similar to those observed with infected BHK cells. At 12 hpi, a few bright foci of TIA-1 that colocalized with WNV proteins were observed, similar to what was observed by 12 hpi for TIAR in BHK cells. By 24 hpi, a significant increase in TIA-1 in the perinuclear region and strong colocalization with viral proteins was observed. At all times, a significant portion of TIA-1 remained in the nucleus. These results indicate that in the absence of TIAR, colocalization of TIA-1 with the viral proteins occurred more quickly with a time course similar to that of TIAR colocalization in cells expressing both proteins.

To determine whether TIA-1 and TIAR colocalization with viral proteins was also characteristic of other flavivirus infections, BHK cells were infected with DV, (MOI of 0.1), a divergent flavivirus from a different serocomplex than WNV, and analyzed at 12, 24, 36, and 72 hpi. Due to the lower MOI, at 12 and 24 hpi, DV proteins were detected in only about 5% of the cells. By 36 hpi, clusters of infected cells with bright virus-specific cytoplasmic foci were observed (−40% of the cells). At 72 hpi, in about 60% of the cells, DV proteins were detected with a distribution and staining intensity similar to that observed in WNV infected cells at 24 hpi. Strong colocalization of both TIAR and TIA-1 with DV proteins was detected at 36 and 72 hpi. This colocalization was primarily in the perinuclear region but was also observed in foci extending into the cytoplasm of the infected cells. In the uninfected cells, TIA-1 and TIAR were equally distributed in the cytoplasm and nucleus. These results suggest that the colocalization of TIA-1 and TIAR with viral proteins is a general feature of flavivirus infections.

### Example 11

### TIA-1 and TIAR Interact with Sites of WNV and DV RNA Replication

In previous in vitro RNA-protein interaction assays, the TIA-1 and TIAR proteins were shown to bind specifically to the WNV3′(−)SL RNA. The minus-strand RNA of flaviviruses is present in infected cells only in RNA replication complexes. An antibody to dsRNA that does not detect either cellular ribosomal RNA or tRNA was previously utilized to detect flavivirus replication complexes in infected cells. To test whether TIA-1 and TIAR colocalized with viral dsRNA in infected cells, BHK cells infected with WNV (MOI of 0.1) or DV (MOI of 0.1) were fixed and incubated with anti-dsRNA and anti-TIA-1 or anti-TIAR antibody. A lower MOI for WNV and later time points were used to better visualize the replication complexes. Perinuclear foci stained with anti-dsRNA antibody were observed in WNV infected cells at 36 hpi but not in mock infected cells. In WNV infected cells, strong colocalization of TIA-1/TIAR with the dsRNA was observed in bright perinuclear foci. Similar dsRNA perinuclear foci and similar TIA-1 and TIAR colocalization with these foci were observed in DV infected cells at 72 hpi, indicating that TIA-1/TIAR strongly colocalized with flavivirus RNA complexes in infected cells.

Previous immunofluorescence studies using antibodies specific to dsRNA as well as to individual flavivirus NS proteins suggested that NS3 and NS5 as well as NS1, NS2a, and NS4a associate with ds viral RNA in flavivirus replication complexes located in the perinuclear region of infected cells. Broader areas of colocalization were observed with the anti-WNV than with the anti-dsRNA antibody, suggesting that TIA-1 and TIAR might also interact with a viral nonstructural protein. Communoprecipitation experiments were done using lysates prepared from mock infected or WNV infected BHK cells at 24 hpi and anti-TIAR or anti-TIA-1 antibody. Cell lysates as well as immunoprecipitates were then analyzed by SDS-PAGE and transferred to nitrocellulose membranes for Western blot analysis using anti-WNV (FIG. 9). Four WNV proteins, presumed to be NS5, NS3, E, and NS1, were detected in lysates from WNV infected cells (FIG. 9, left panel, lane 2), but not in lysates or immunoprecipitates from mock infected cells (FIG. 9, left panel, lanes 1, 3, and 5). In immunoprecipitates from WNV infected cells, both anti-TIAR (FIG. 9, left panel, lane 4) and anti-TIA-1 (FIG. 9, left panel, lane 6) antibody co-immunoprecipitated a viral protein with the expected size of NS3. To verify that this was NS3, samples precipitated with anti-TIA-1 or anti-TIAR antibodies was analyzed by Western blot analysis using an anti-WNV NS3 monoclonal antibody. NS3 was detected in lysates from WNV infected cells after immunoprecipitation with both anti-TIAR (FIG. 9, right panel, lane 4) or anti-TIA-1 (FIG. 9, right panel, lane 6) antibody. In FIG. 9, BHK cells were infected with WNV at a MOI of 0.1. Laser scanning confocal microscopy of mock infected and WNV infected cells stained with anti-dsRNA or anti-NS3 and then with either anti-TIAR or anti-TIA-1 antibodies at 36 hpi. Communoprecipitation of WNV proteins by anti-TIA-1 and anti-TIAR antibodies was performed in BHK cells, which were infected with WNV at a MOI of 5. Immunoprecipitates were visualized by Western blot using anti-WNV (left panel) or anti-NS3 antibody (right panel). Lane 1, Mock infected lysate; Lane 2, WNV infected lysate; Lanes 3 and 5, Mock infected immunoprecipitates; Lanes 4 and 6, WNV infected immunoprecipitates. Arrows indicate the positions of the WNV proteins detected. Positions of molecular weight standards are indicated on the left.

Anti-WNV NS3 monoclonal antibody was next used to study the colocalization of TIA-1/TIAR with NS3 in BHK cells infected with WNV or DV at 36 or 72 hpi, respectively. NS3 was previously shown to be broadly distributed around the nucleus and to colocalize with dsRNA foci in flavivirus-infected cells. In the merged images, both TIA-1 and TIAR strongly colocalized with NS3 in WNV infected cells. Bright focal areas as well as more diffuse areas of colocalization were observed. Similar results were observed in DV infected cells at 72 hpi.

### Example 12

### WNV and DV Interfere with Stress Granule Formation in Infected BHK Cells

TIA-1/TIAR were previously reported to be required for the formation of stress granules in cells after treatment with arsenite and both proteins were shown to colocalize with viral nonstructural proteins and replication complexes in flavivirus infected cells. To determine whether a flavivirus infection would interfere with cellular stress granule formation, BHK cells were mock infected or infected with WNV and exposed to oxidative stress by treatment with 0.5 mM arsenite for 30 min. Infected cells were treated at 6, 12, or 24 hpi (hours post-infection). The cells were then fixed and stained with anti-WNV and anti-TIAR antibody. As described previously, at 6 hpi, only a small amount of colocalization of TIAR with WNV proteins was observed. The number of stress granule induced by the arsenite was slightly reduced (FIG. 10A) as compared to mock infected cells (FIG. 10A). At 12 hpi, stress granule formation further decreased (FIG. 10A) as colocalization of TIAR and WNV proteins increased. Few if any stress granules were detected at 24 hpi (FIG. 10A) in cells showing strong colocalization between TIAR and WNV proteins. WNV infection alone did not induce stress granule formation at any time after infection. The effect of DV infection on stress granule formation was also investigated. BHK cells infected with DV for 36 or 72 hr were treated with 0.5 mM arsenite for 30 min and immunostained with anti-DV and anti-TIAR. At both times, strong colocalization of TIAR and DV proteins was observed. Few stress granules were observed in infected cells (FIG. 12A), whereas many stress granule were observed in uninfected cells. Laser scanning confocal microscopy of mock infected and WNV infected (MOI of 5) BHK cells treated with 0.5 mM arsenite for 30 min at the indicated times after infection was performed. The cells were fixed, permeabilized, and stained with anti-WNV and with the SG marker, anti-TIAR. In FIG. 10A, quantification of the number of SG in mock-infected (M) cells and WNV-infected cells at the indicated times after infection was performed. Stress granules (SG) in 70 cells were counted for each experimental condition and the average number of SG/cell was plotted. Error bars indicate the standard deviation of the mean. FIG. 10B shows a Western blot analysis of phospho-eIF2α in BHK cells mock infected or infected with WNV (MOI of 5) and then treated or not treated with 0.5 mM of arsenite for 30 min at the indicated times after infection. Lane 1, Mock infected BHK cells; Lane 2, cells treated with 0.5 mM arsenite for 30 min; Lane 3, cells infected with WNV; Lanes 4-6, cells infected with WNV and then treated with arsenite for 30 min at the indicated times. Phospho-eIF2α (S51) (upper panel), total eIF2α (middle panel), and G3BP (lower panel) were detected with specific antibodies.

Since phosphorylation of eIF2α at S51 also regulates stress granule formation, the effect of WNV and DV infections on eIF2α phosphorylation was assayed. BHK cells were infected with either virus and at various times after infection, cells were treated for 30 min with 0.5 mM arsenite. Control infected cells were not treated with arsenite. Proteins in cell lysates were analyzed by Western blot using a polyclonal antibody specific for eIF2α phosphorylated at S51 (FIG. 10B). No eIF2α phosphorylation was detected in mock infected cells or in cells infected with WNV (FIG. 10B, lane 3) or DV (FIG. 12B, lane 1) at any time after infection. High levels of eIF2α phosphorylation were observed in extracts of mock infected cells treated with arsenite (FIG. 10B, lane 2). Reduced levels of phospho-eIF2α were detected in WNV infected extracts by 12 hpi and were further decreased by 24 hpi (FIG. 10B, lanes 4-6). In the DV extracts, decreased phosphorylation of eIF2α was observed at later times post infection (FIG. 12B, lanes 2-5). The large number of uninfected cells present in the DV infected cultures are the source of the higher levels of phospho-eIF2α observed until 6 dpi. The levels of total eIF2α and of another protein involved in stress granule formation, G3BP, remained constant under all conditions tested and after infection with both viruses (FIGS. 10B and 12B). Although neither WNV nor DV infection induced stress granule formation in BHK cells, cells infected with both flaviviruses became progressively more resistant to stress granule induction by arsenite with time after infection. In FIG. 12 A, laser scanning confocal microscopy of mock infected and DV infected (MOI of 0.1) BHK cells treated with 0.5 mM arsenite for 30 min at the indicated times was performed. The cells were fixed, permeabilized, and stained with anti-DV and with the SG marker, anti-TIAR. Quantification of SG in mock-infected cells (M) and DV-infected cells was performed at different times after infection (36 and 72 hpi). The number of stress granules in 70 cells for each experimental condition was counted and the average number of SG/cell was plotted. Error bars indicate the standard deviation of the mean. In FIG. 10B, Western blot analysis of phospho-eIF2α in BHK cells mock infected or infected with DV and then treated or not treated with of arsenite for 30 min at various times after infection was performed. Lane 1, cells infected with DV2 (MOI of 0.1); Lanes 2-5, cells infected with DV2 and then treated with arsenite at the indicated times post infection. Phospho-eIF2α (S51) (upper panel), total eIF2α (middle panel), and G3BP (lower panel) were detected with specific antibodies.

### Example 13

### WNV and DV Infections Interfere with Processing Body Assembly

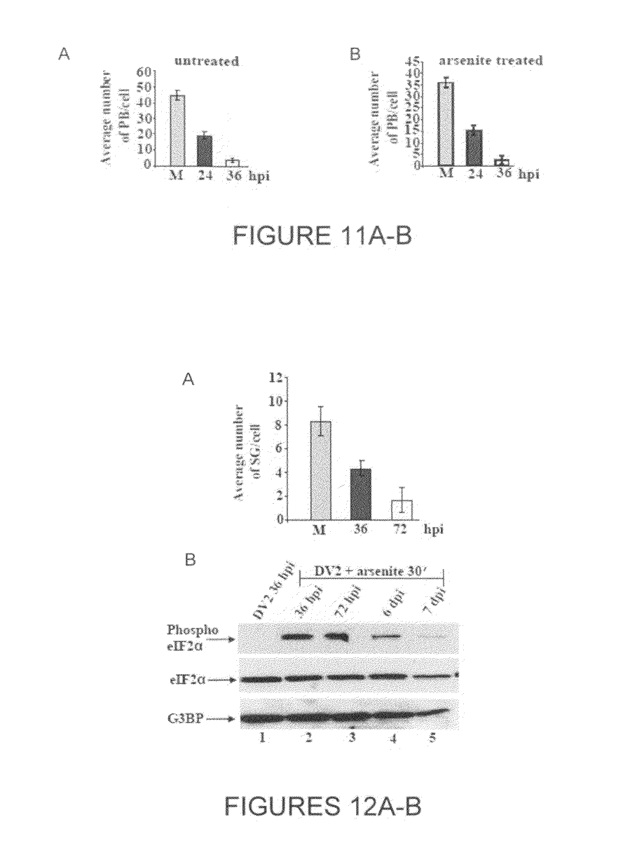

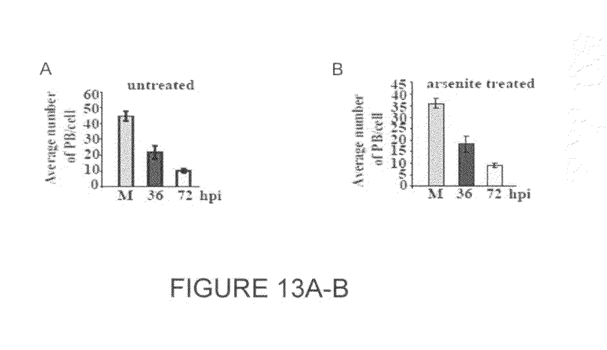

Stress granules and processing bodies share some components and are dynamically linked via their role in regulating cytoplasmic mRNA storage and/or degradation. To test whether WNV and DV infections also interfere with processing body assembly, BHK cells mock infected or infected with either WNV or DV were treated or not treated for 30 min with 0.5 mM arsenite at various times after infection. The cells were then fixed and incubated with antibody to the processing body marker DCP1a and anti-WNV or anti-DV. Processing bodies were detected in untreated cells as well as in arsenite treated cells. In uninfected, WNV infected, and DV infected cells, processing bodies were evenly distributed throughout the cytoplasm. In both uninfected and infected cells treated with arsenite, the number of processing bodies was decreased (FIG. 11; FIG. 13) and the remaining processing bodies were mainly located around the nucleus. In WNV infected cells, a noticeable reduction in processing body formation were observed by 24 hpi in untreated (FIG. 11A) and arsenite-treated (FIG. 11B) cells as compared to mock infected cells (FIG. 11). A further reduction in processing body formation was observed in both arsenite-untreated (FIG. 11A) and treated (FIG. 11B) WNV-infected cells at 36 hpi. In DV infected cells, a reduction in processing body formation was observed at later times post infection (FIGS. 13A and 13B) consistent with the slower replication kinetics of DV. These results indicate that both WNV and DV infections interfere with processing body assembly in BHK cells. Laser scanning confocal microscopy of mock infected and WNV infected (MOI of 5) cells that were treated or not treated with arsenite for 30 min at the indicated times was performed. The cells were stained with anti-WNV antibody and then with anti-DCP1a (a PB marker) antibody. Quantification of the number of PB in mock-infected cells (M) and WNV-infected cells treated (FIG. 11A) or not treated (FIG. 11B) with arsenite. Processing bodies in 50 cells were counted for each experimental condition at the indicated times after infection and the average number of PB/cell was plotted. Error bars indicate the standard deviation of the mean. In FIG. 13, laser scanning confocal microscopy of mock infected and DV infected (MOI of 0.1) cells that were not treated or treated with arsenite for 30 min at the indicated times after infection was performed. The cells were then stained with anti-DV2 and anti-DCP1a (a PB marker) antibody. Quantification of PB in mock-infected cells (M) and DV2-infected cells treated (right) or not treated (left) with arsenite was performed. Processing bodies were counted in 50 cells under each experimental condition at the indicated times and the average number of PB/cell was plotted. Error bars indicate the standard deviation of the mean.

What is claimed is:

1. A method of inhibiting replication of a flavivirus, comprising,
administering an effective amount of a composition; and
inhibiting flavivirus replication by inhibiting or interfering with at least one host cell protein associated with replication of a flavivirus

2. The method of claim 1, wherein the flavivirus is West Nile virus.

3. The method of claim 1, wherein the flavivirus is Dengue virus.

4. The method of claim 1, wherein the composition comprises a nucleic acid construct of the 3′ end of the negative strand nucleic acid of a flavivirus or a nucleic acid construct that mimics the 3′ end of the negative strand nucleic acid of a flavivirus.

5. The method of claim 1, wherein the composition comprises a peptide that mimics a region of at least one viral protein that interacts with at least one host cell protein.

6. The method of claim 1, wherein the composition comprises a compound that interferes with at least one host cell protein associated with replication of a flavivirus.

7. The method of claim 1, wherein the at least one host cell protein is TIAR, TIA-1, p50, p60, p108, p52, p84, or p105.

8. A method of inhibiting replication of a flavivirus, comprising,
administering an effective amount of a composition; and
inhibiting flavivirus replication by inhibiting or interfering with the interaction or association of at least one host cell protein and at least one viral protein.

9. The method of claim 8, wherein the flavivirus is West Nile virus.

10. The method of claim 8, wherein the composition comprises a protein, polypeptide, peptide, or amino acid sequence that mimics a region of the at least one viral protein that interacts with at least one host cell protein.

11. The method of claim 9, wherein the at least one viral protein is the capsid protein, membrane protein, envelope protein, NS1, NS2a, NS2b, NS3, NS4a, NS4b, or NS5 of West Nile virus.

12. The method of claim 8, wherein the flavivirus is Dengue virus.

13. The method of claim 8, wherein the at least one viral protein is a structural or non-structural protein of Dengue virus.

14. The method of claim 8, wherein the at least one host cell protein is TIA-1, TIAR p50, p60, p108, p52, p84, or p105.

15. A method of inhibiting replication of a flavivirus, comprising,
administering an effective amount of a composition; and
inhibiting flavivirus replication by inhibiting or interfering with a host cell signaling pathway.

16. The method of claim 15, wherein the composition comprises double-stranded RNA, interferons, viruses, replication-deficient viruses, small molecules, small organic molecules, cytokines, chemokines, viral vectors, proteins, peptides, peptide agonists, growth factors, nucleic acid, antibodies, peptoids, polyanions, or other cell signaling proteins or molecules.

17. The method of claim 15, wherein the host cell signaling pathway comprises eIF2α or PKR


SEQUENCE LISTING (2 sequences)

SEQ ID NO: 1 - DNA, 21 bp, Inovirus C2
caggaaacagctatgaccatg

SEQ ID NO: 2 - DNA, 20 bp, Inovirus C2
agtagttcgcctgtgtgagc